(19) United States
(12) Patent Application Publication
(10) Pub. No.: US 2022/0400949 A1
(43) Pub. Date: Dec. 22, 2022

(54) SLIT-LAMP MICROSCOPE

(71) Applicant: Bolan, LLC, Troy, MI (US)

(72) Inventors: Huanlan Lin, Shanghai (CN); Le Lin, Troy, MI (US)

(21) Appl. No.: 17/350,540

(22) Filed: Jun. 17, 2021

(51) Int. Cl. A61B 3/135; A61B 3/00; A61B 3/14

(52) U.S. Cl. CPC A61B 3/135; A61B 3/0008; A61B 3/14

(57) ABSTRACT

A slit-lamp microscope includes a base plate defining an x-axis and a z-axis. The microscope includes a movable base member slidably disposed on the base plate. The microscope includes a computer that generates first and second control signals to induce first and second movement devices, respectively, to move the movable base member in response to a first x-axis command and a first z-axis command, respectively, in first and second remote control messages, respectively. The computer generates a third control signal to induce the digital camera of the microscope assembly to generate a digital image from the light received from the subjects eye in response to the photograph command in a third remote control message.

### BACKGROUND

The inventors herein have recognized a need for a slit-lamp microscope that is remotely controllable utilizing remote control messages from a remote device. In particular, the inventors herein have recognized that it would be desirable to have a slit-lamp microscope with first, second, and third movement devices that can move a movable base member parallel to an x-axis, a y-axis, and a z-axis, respectively in response to remote control messages received by a remote communication module in the slit-lamp microscope. Further, the inventors have recognized that it would be desirable for the slit-lamp microscope to have a slit size adjustment device to move first and second blades, respectively of a slit forming device in response to a remote control message received by the remote communication module.

### SUMMARY

A slit-lamp microscope in accordance with an exemplary embodiment is provided. The slit-lamp microscope includes a base plate defining an x-axis and a z-axis. The slit-lamp microscope further includes a movable base member slidably disposed on the base plate. The slit-lamp microscope further includes a first movement device operably coupled to the base plate and the movable base member. The first movement device moves the movable base member left and right parallel to the x-axis. The slit-lamp microscope further includes a second movement device operably coupled to the base plate and the movable base member. The second movement device moves the movable base member forward and backward parallel to the z-axis. The slit-lamp microscope further includes a lower chassis operably coupled to the movable base member. The slit-lamp microscope further includes a first support arm that is coupled to the lower chassis and to a microscope assembly. The slit-lamp microscope further includes a second support arm being coupled to the lower chassis and an illumination assembly. The illumination assembly has a slit forming device and a light source device. The slit forming device has a slit for receiving light therethrough that is output from the light source device, and projects the light from the slit forming device onto a subjects eye. The microscope assembly receives light from the subjects eye. The microscope assembly has a digital camera therein. The slit-lamp microscope further includes a computer operably coupled to the first, second, and third movement devices, the microscope assembly, and a remote communication module. The remote communication module receives first, second, and third remote control messages with a first x-axis command, a first z-axis command, and a photograph command, respectively, and the remote communication module sends the first x-axis command, the first z-axis command, and the photograph command to the computer. The computer generates the first and second control signals to induce the first and second movement devices, respectively, to move the movable base member in response to the first x-axis command and the first z-axis command, respectively. The computer generates a third control signal to induce the digital camera of the microscope assembly to generate a digital image from the light received from the subjects eye in response to the photograph command.

A slit-lamp microscope in accordance with another exemplary embodiment is provided. The slit-lamp microscope includes a base plate. The slit-lamp microscope further includes a movable base member slidably disposed on the base plate. The slit-lamp microscope further includes a lower chassis operably coupled to the movable base member. The slit-lamp microscope further includes a first support arm that is coupled to the lower chassis and to a microscope assembly. The slit-lamp microscope further includes a second support arm that is coupled to the lower chassis and an illumination assembly. The illumination assembly has a slit forming device and a light source device. The slit forming device has a slit for receiving light therethrough that is output from the light source device, and projects the light from the slit forming device onto a subjects eye. The microscope assembly receives light from the subjects eye. The microscope assembly has a digital camera therein. The slit-lamp microscope further includes a computer operably coupled to the slit size adjustment device, the microscope assembly, and a remote communication module. The remote communication module receives first and second remote control messages having a slit size command and a photograph command, respectively, and the remote communication module sends the slit size command and the photograph command to the computer. The computer generates a first control signal to induce the slit size adjustment device to move first and second blades, respectively, of the slit forming device to obtain a desired size of the slit in response to the slit size command. The computer generates a second control signal to induce the digital camera in the microscope assembly to generate a digital image from the light received from the subjects eye in response to the photograph command.

### BRIEF DESCRIPTION OF THE DRAWINGS

FIG. 1 is an isometric view of a slit-lamp microscope system having a slit-lamp microscope in accordance with an exemplary embodiment, a communication network, and a remote computer;

FIG. 2 is an isometric view of the slit-lamp microscope of FIG. 1 in accordance with an exemplary embodiment;

FIG. 3 is a block diagram of components of the slit-lamp microscope system of FIG. 1;

FIG. 4 is another isometric view of the slit-lamp microscope of FIG. 1 having outer cover portions removed therefrom;

FIG. 5 is a front view of the slit-lamp microscope of FIG. 1;

FIG. 6 is a side view of the slit-lamp microscope of FIG. 1 having outer cover portions removed therefrom;

FIG. 7 is another side view of the slit-lamp microscope of FIG. 1 having outer cover portions removed therefrom;

FIG. 8 is another isometric view of the slit-lamp microscope of FIG. 4;

FIG. 9 is another isometric view of the slit-lamp microscope of FIG. 4;

FIG. 10 is another isometric view of the slit-lamp microscope of FIG. 4;

FIG. 11 is a cross-sectional schematic of an illumination assembly of FIG. 6 taken along lines 11-11 in FIG. 6;

FIG. 12 is a top view of the slit-lamp microscope of FIG. 4;

FIG. 13 is a schematic of optical components utilized in the slit-lamp microscope of FIG. 1;

FIG. 14 is a partially exploded view of a base plate, a movable base member, and a second movement device utilized in the slit-lamp microscope of FIG. 1;

FIG. 15 is a top view of the base plate, the movable base member, and a second movement device utilized in the slit-lamp microscope of FIG. 1;

FIG. 16 is a partial exploded view of a base plate, a movable base member, and a first movement device utilized in the slit-lamp microscope of FIG. 1;

FIG. 17 is a cross-sectional view of a portion of the movable base member and the first movement device of FIG. 16 taken along lines 17-17 in FIG. 16;

FIG. 18 is partial exploded view of a portion of the slit-lamp microscope of FIG. 1;

FIG. 19 is a partial exploded view of a movable base member utilized in the slit-lamp microscope of FIG. 1;

FIG. 20 is another partial exploded view of the movable base member of FIG. 19;

FIG. 21 is another partial exploded view of the movable base member of FIG. 19;

FIG. 22 is a partial exploded view of a portion of the slit-lamp microscope of FIG. 1;

FIG. 23 is a partial exploded view of a base plate, a movable base member, a lower chassis, first and second support arms, and an illumination assembly utilized in the slit-lamp microscope of FIG. 1;

FIG. 24 is an isometric view of a portion of the illumination assembly utilized in the slit-lamp microscope of FIG. 1;

FIG. 25 is an isometric view of a portion of the illumination assembly of FIG. 23;

FIG. 26 is another isometric view of a portion of the illumination assembly of FIG. 23;

FIG. 27 is an isometric view of a slit forming device utilized in the illumination assembly of FIG. 24;

FIG. 28 is another isometric view of the slit forming device of FIG. 27;

FIG. 29 is another isometric view of the slit forming device of FIG. 27;

FIG. 30 is a partial exploded view of the illumination assembly of FIG. 23;

FIG. 31 an isometric view of the slit forming device utilized in the illumination assembly of FIG. 30;

FIG. 32 is another isometric view of the slit forming device of FIG. 30;

FIG. 33 is another isometric view of the slit forming device of FIG. 30;

FIG. 34 is another isometric view of the slit forming device of FIG. 30;

FIG. 35 is another isometric view of the slit forming device of FIG. 30;

FIG. 36 is a side view of the slit forming device of FIG. 30;

FIG. 37 is an isometric view of the illumination assembly utilized in the slit-lamp microscope of FIG. 1;

FIG. 38 is a partial exploded view of the illumination assembly of FIG. 37;

FIG. 39 is a partial exploded isometric view of the illumination assembly and a second support arm utilized in the slit-lamp microscope of FIG. 1;

FIG. 40 is another partial exploded isometric view of the illumination assembly and the second support arm of FIG. 39;

FIG. 41 is a cross-sectional schematic of the illumination assembly of FIG. 37 taken along lines 41-41 in FIG. 37;

FIG. 42 is a partial exploded view of a top portion of the illumination assembly of FIG. 37;

FIG. 43 is another partial exploded view of the top portion of the illumination assembly of FIG. 37;

FIG. 44 is an isometric view of the top portion of the illumination assembly of FIG. 37;

FIG. 45 is a partial exploded view of a microscope assembly utilized in the slit-lamp microscope of FIG. 1;

FIG. 46 is a partial exploded view of a portion of the microscope assembly of FIG. 45;

FIG. 47 is a partial exploded view of a chin rest assembly utilized in the slit-lamp microscope of FIG. 1;

FIG. 48 is a cross-sectional schematic of the chin rest assembly of FIG. 47 taken along lines 47-47 in FIG. 47; and

FIGS. 49-52 are flowcharts of a method for operating the slit-lamp microscope system of FIG. 1 in accordance with another exemplary embodiment.

### DETAILED DESCRIPTION

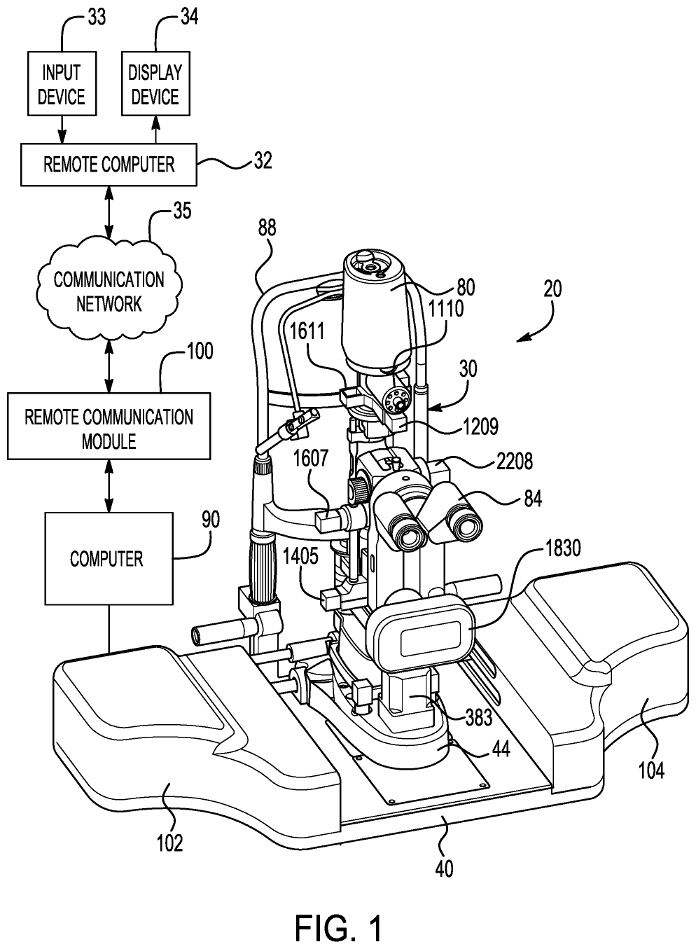

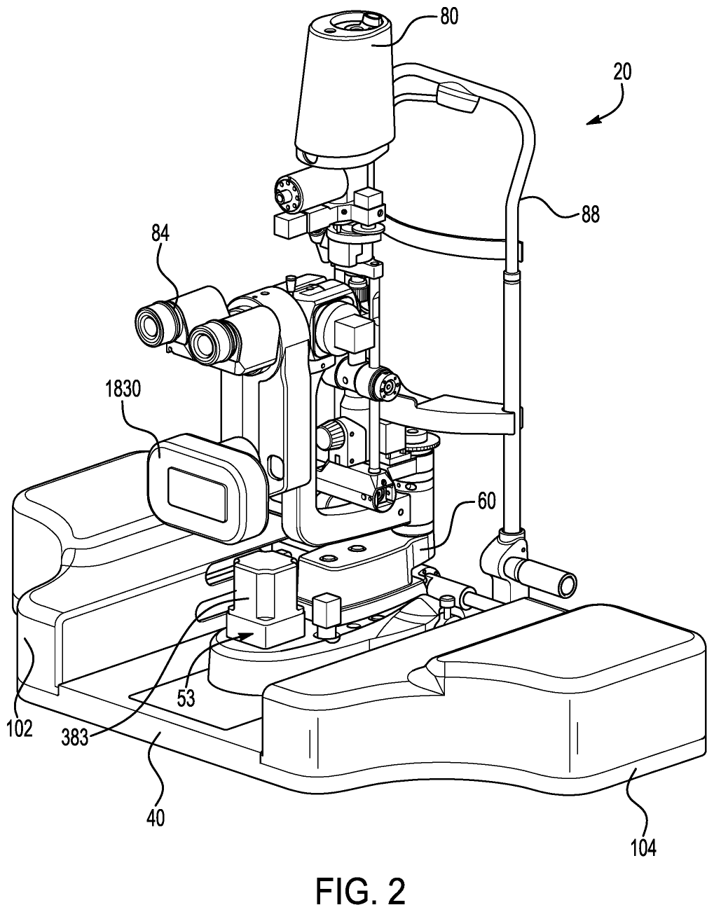

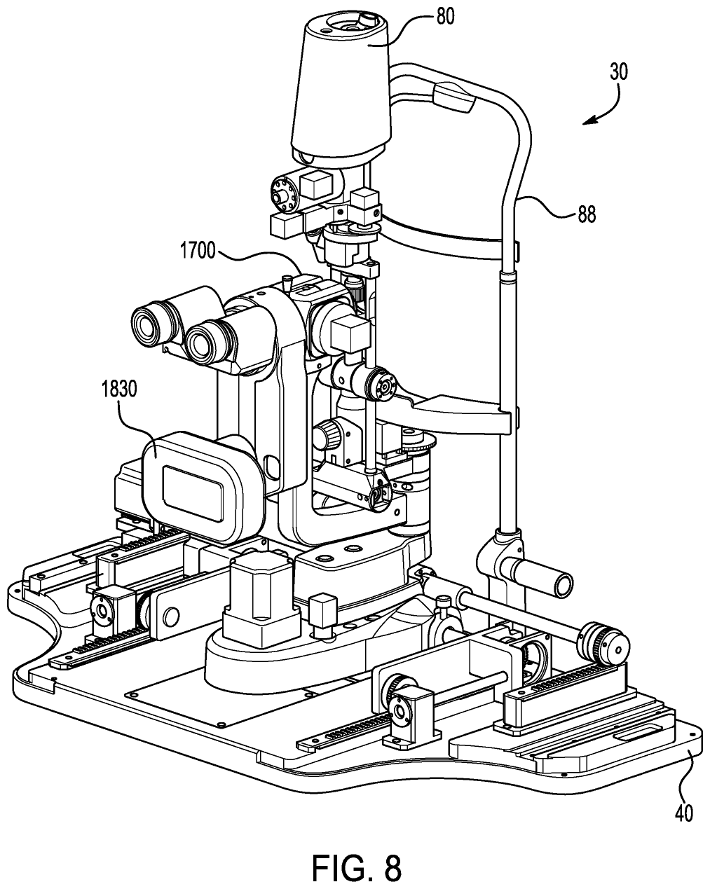

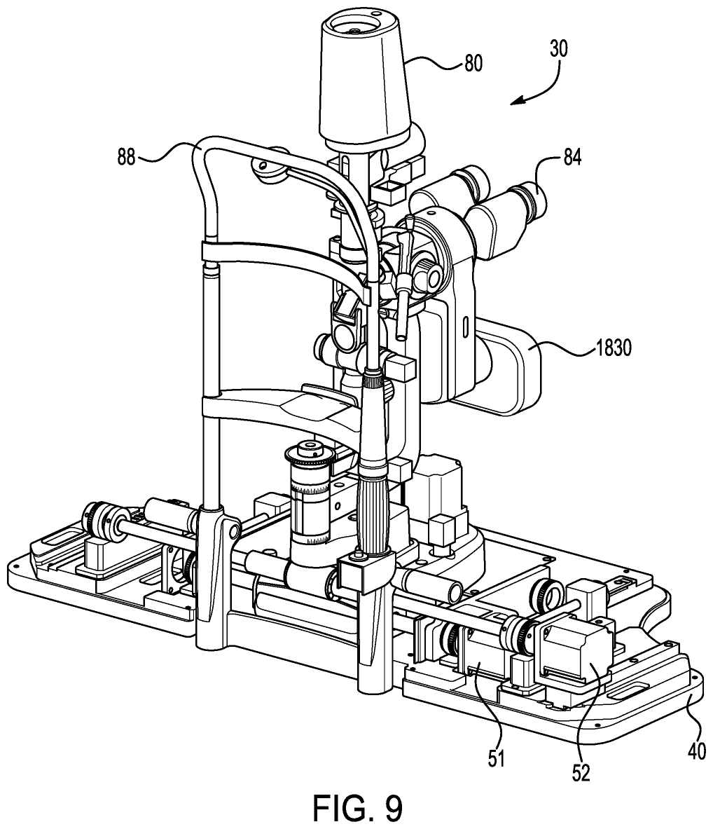

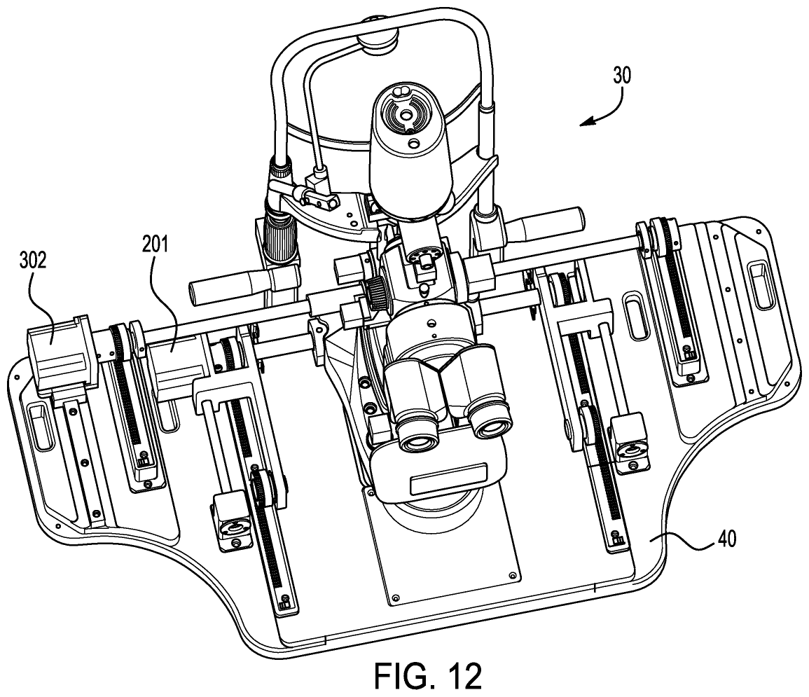

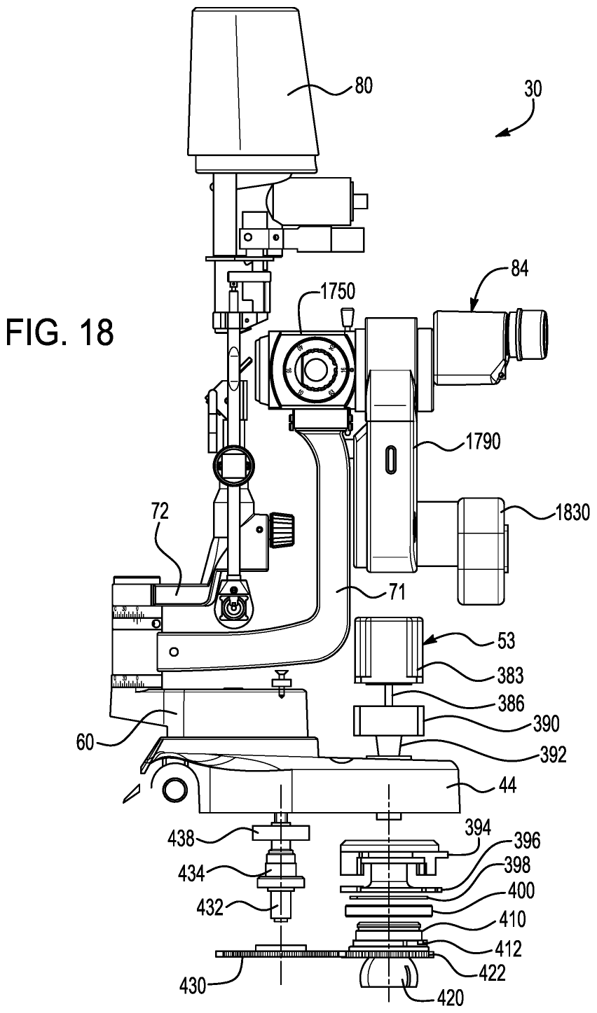

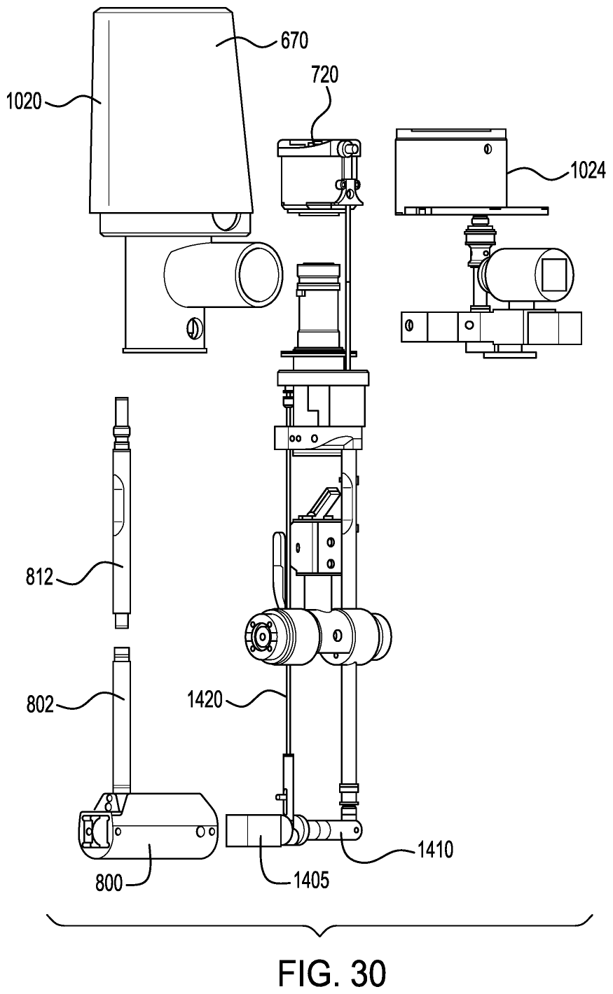

Referring to FIGS. 1-48, a slit-lamp microscope system 20 having a slip-lamp microscope 30 in accordance with an exemplary embodiment, a remote computer 32, an input device 33, a display device 34, and a communication network 35 are illustrated.

Before describing the system 20 in greater detail, a few terms utilized herein will be described.

The term “remote control message” is a message is that transmitted from a computer that is remote (i.e., in a different physical facility or building) than the slit-lamp microscope 20. In an exemplary embodiment, the remote control message is a message that is transmitted through a communication network such as the Internet for example. Further, the remote control message can be transmitted through electrical lines or transmitted wirelessly using radio-frequency (RF) signals.

Referring to FIGS. 1-12, the slit-lamp microscope 30 includes a base plate 40, a movable base member 44, a first movement device 51, a second movement device 52, a third movement device 53, a lower chassis 60, a first support arm 71, a second support arm 72, an illumination assembly 80, a microscope assembly 84, a chin rest assembly 88, a computer 90, a remote communication module 100, and outer cover portions 102, 104.

Base Plate

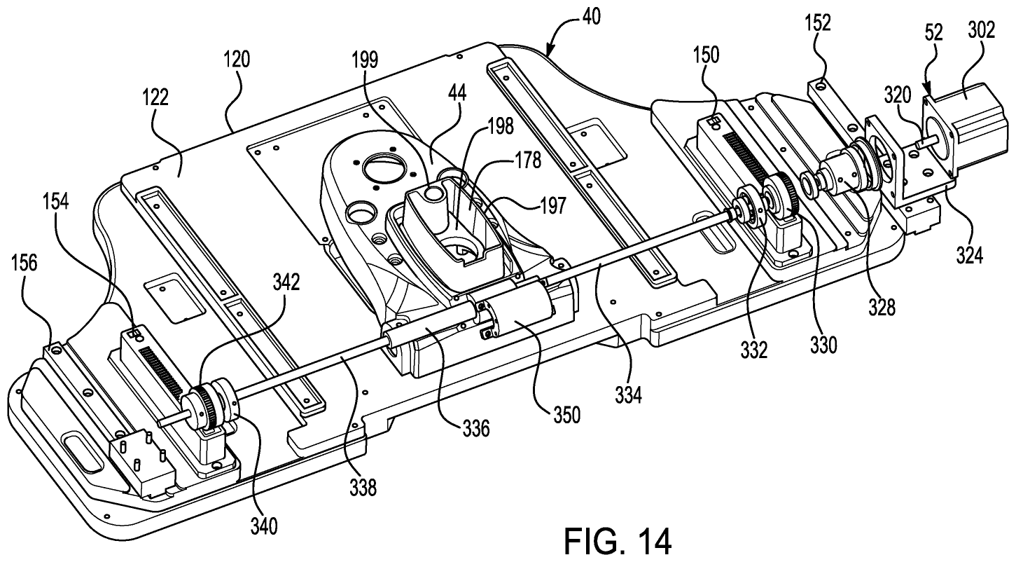

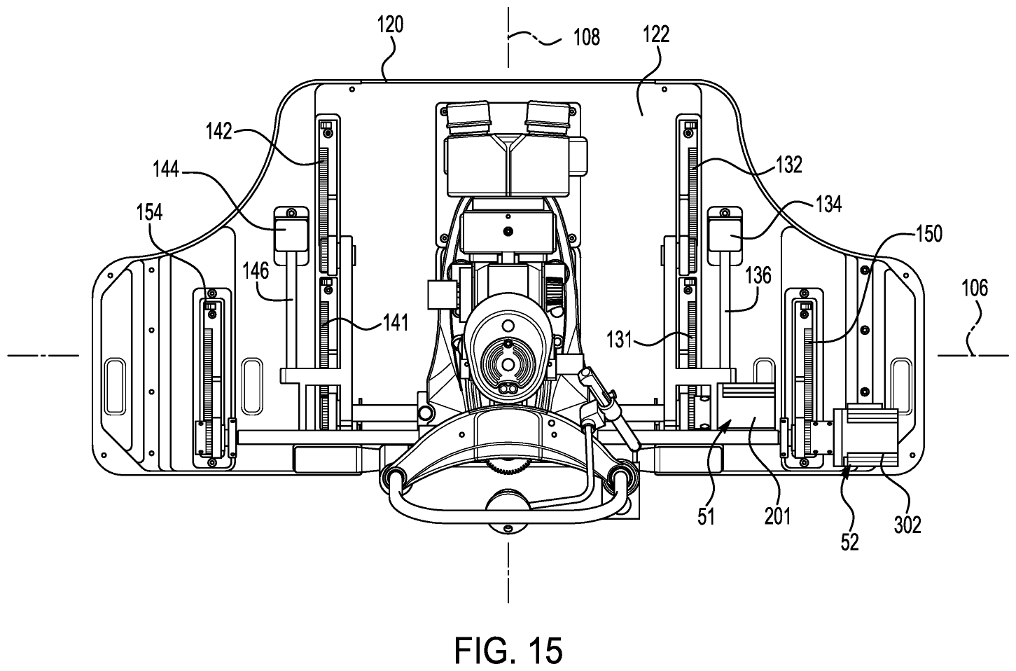

Referring to FIGS. 1 and 14-17, the base plate 40 is provided to hold the remaining components of the slit-lamp microscope 30 thereon. The base plate 40 includes a plate portion 120, rack members 131, 132 (shown in FIG. 15), a stationary bracket 134, a guide shaft 136, rack members 141, 142, a stationary bracket 144, a guide shaft 146, a rack member 150 (shown in FIG. 14), a slider bar 152, a rack member 154, and a slider bar 156.

The plate portion 120 includes a top surface 122. The remaining components of the base plate 40 are coupled to the top surface 122. In an exemplary embodiment, the plate portion 120 is constructed of a metal such as steel or aluminum.

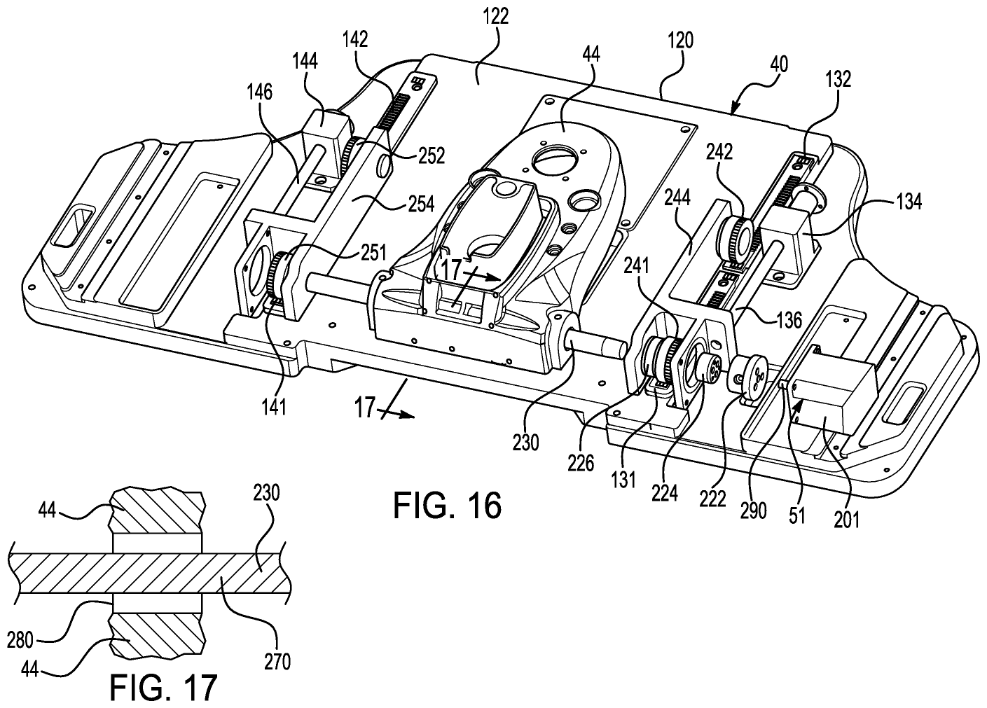

Referring to FIGS. 15 and 16, the rack members 131, 132 are coupled to the top surface 122 of the plate portion 120. The rack members 131, 132 are disposed parallel to a z-axis (shown in FIG. 15) of the base plate 40 and are aligned with one another. The rack members 131, 132 are provided to rotatably support the pinion gears 241, 242 (shown in FIG. 16) of the first movement device 51. The first movement device 51 moves the movable base member 44 in a direction parallel to the x-axis (shown in FIG. 15) as will be described in greater detail below.

The stationary bracket 134 is coupled to the top surface 122 of the plate portion 120 and is provided to slidably support the guide shaft 136. In particular, the guide shaft 136 slides in a direction disposed parallel to the z-axis within the stationary bracket 134.

The rack members 141, 142 are coupled to the top surface 122 of the plate portion 120. The rack members 141, 142 are disposed parallel to the z-axis (shown in FIG. 15) of the base plate 40 and are aligned with one another. The rack members 141, 142 are provided to rotatably support the pinion gears 251, 252 of the first movement device 51. The first movement device 51 moves the movable base member 44 in a direction parallel to the x-axis (shown in FIG. 15) as will be described in greater detail below.

The stationary bracket 144 is coupled to the top surface 122 of the plate portion 120 and is provided to slidably support the guide shaft 146. In particular, the guide shaft 146 slides in a direction parallel to the z-axis within the stationary bracket 144.

Referring to FIGS. 14 and 15, the rack members 150, 154 are coupled to the top surface 122 of the plate portion 120. The rack members 150, 154 are disposed parallel to a z-axis (shown in FIG. 15) of the base plate 40 and apart from one another. The rack member 150 is provided to rotatably support the pinion gear 330 of the second movement device 52 that moves the base plate 40 parallel to the z-axis. The rack member 154 is provided to rotatably support the pinion gear 342 of the second movement device 52 that moves the movable base member 44 parallel to the z-axis.

The slider bars 152, 156 are coupled to the top surface 122 of the plate portion 120. The slider bars 152, 156 are disposed parallel to a z-axis (shown in FIG. 15) of the base plate 40 and apart from one another. The slider bar 152 is provided to slidably support the slidable bracket 324 thereon.

Movable Base Member

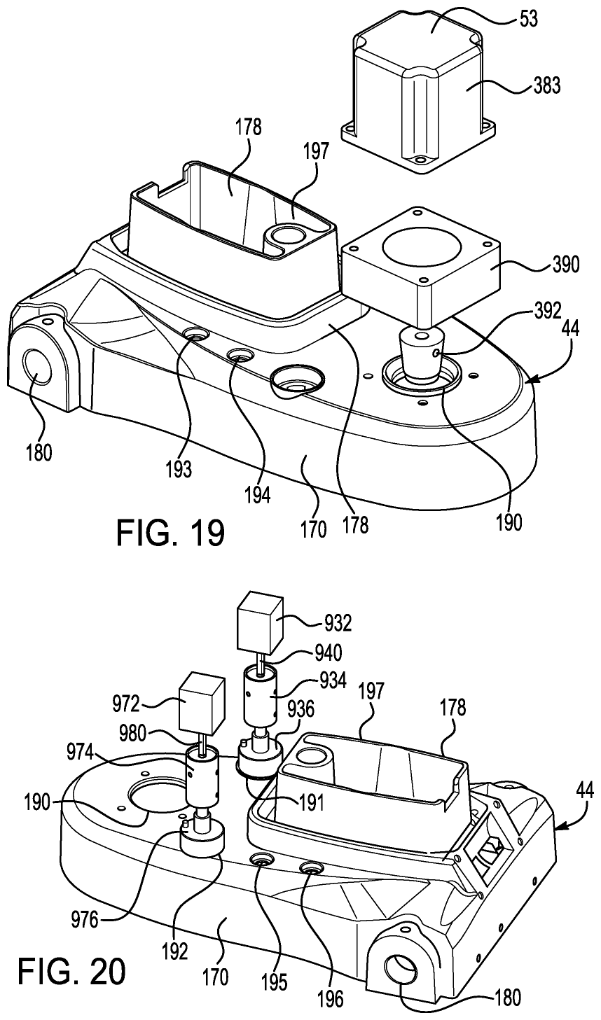

Referring to FIGS. 1, 14, 16, 19 and 20, the movable base member 44 is slidably disposed on the top surface 122 of the base plate 40. The movable base member 44 includes a housing 170 and a chassis receiving portion 178. The housing 170 includes a horizontal aperture 180 extending therethrough for receiving the rotatable shaft 230 (shown in FIG. 16) therethrough. The housing 170 further includes apertures 190, 191, 192, 193, 194, 195, 196 extending therethrough. The aperture 190 receives the connecting shaft 392 (shown in FIG. 19) therethrough. The aperture 191 receives the potentiometer 936 (shown in FIG. 20) and the coupler member 934 therethrough. The aperture 192 receives the potentiometer 976 (shown in FIG. 20) and the coupling member 974 therethrough.

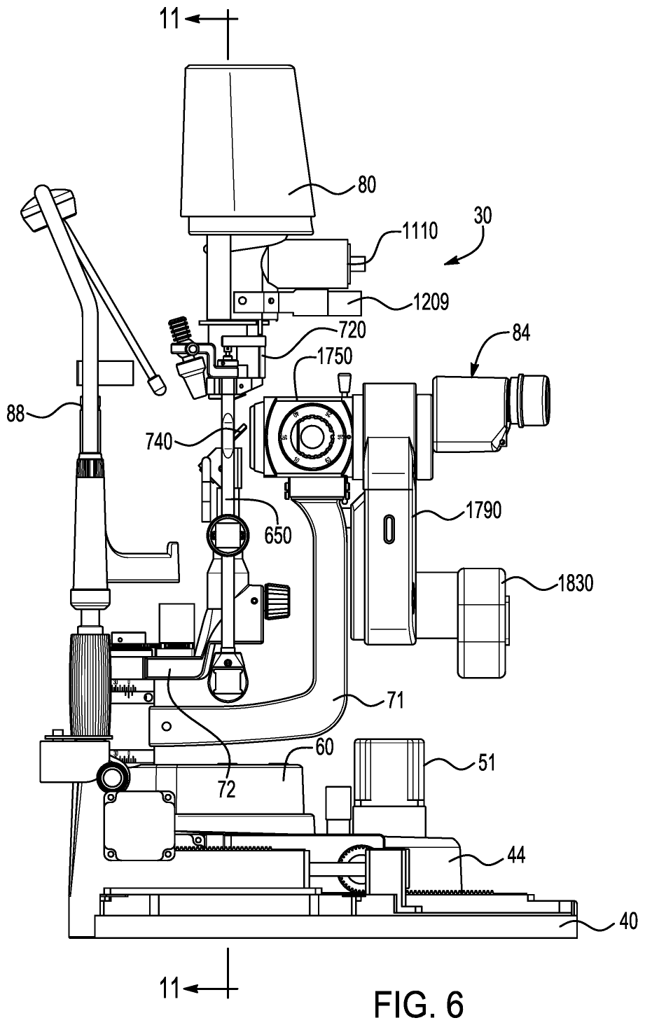

Referring to FIG. 14, the chassis receiving portion 178 includes an outer side wall portion 197, a bottom wall 198, and a screw receiver 199. The bottom wall 198 is coupled to a bottom portion of the outer side wall portion 197. The screw receiver portion 199 is provided to receive a screw for coupling the chassis receiving portion 178 to the lower chassis 60 (shown in FIG. 6).

First Movement Device

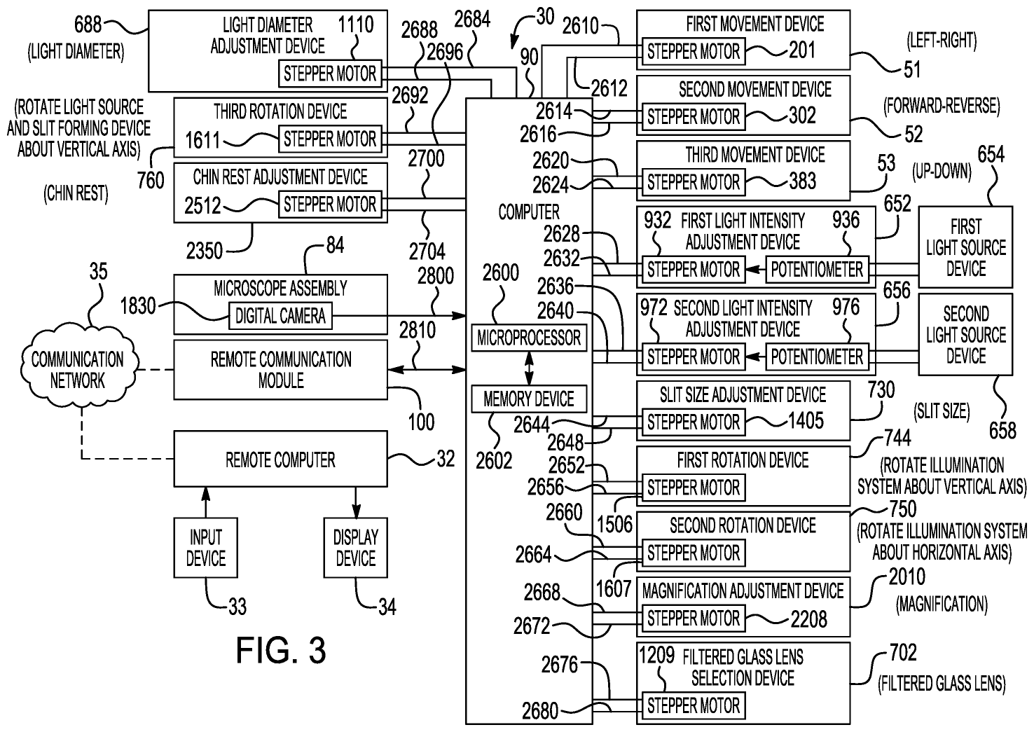

Referring to FIGS. 3, 15, 16 and 17, the first movement device 51 is operably coupled to the base plate 40 and the movable base member 44. The first movement device 51 moves the movable base member left and right parallel to the x-axis 106 (shown in FIG. 15). The first movement device 51 includes a stepper motor 201, a coupling member 222, a coupling member 224, a coupling member 226, a rotatable shaft 230, pinion gears 241, 242, a slidable bracket 244, pinion gears 251, 252, and a slidable bracket 254. The stepper motor 201 has a rotatable shaft 290 (shown in FIG. 16) that rotates in response to a control signal from the computer 90. The stepper motor 201 is supported by the slidable bracket 244. The rotatable shaft 290 is coupled to the coupling member 222 which is further coupled to the coupling member 224. The coupling member 224 is further coupled to the coupling member 226. The pinion gear 131 rotates independently of the coupling members 224, 226 and is rotatably supported by the coupling members 224, 226. The coupling member 226 is further coupled to the rotatable shaft 230. The rotatable shaft 230 extends through an aperture 180 (shown in FIG. 20) in the movable base member 44. The rotatable shaft 230 is further coupled to the pinion gear 141 that rotates independently of the rotatable shaft 230. The rotatable shaft 230 includes a lead screw portion 270 (shown in FIG. 17) and a lead nut 280. The lead nut 280 is coupled to the lead screw portion 270 of the rotatable shaft 230 and is further coupled to the movable base member 44.

During operation, when the stepper motor 201 rotates the rotatable shaft 290 in a first rotational direction, the coupling member 222, the coupling member 224, the coupling member 226, and the rotatable shaft 230 rotate in the first rotational direction such that the lead screw portion 270 urges the lead nut 280 and the movable base member 44 to move in a first direction parallel to the x-axis 106 (shown in FIG. 15) on the top surface 122 of the plate portion 120. Alternately, when the stepper motor 201 rotates the rotatable shaft 290 in a second rotational direction (opposite to the first rotational direction), the coupling member 222, the coupling member 224, the coupling member 226, and the rotatable shaft 230 rotate in the second rotational direction such that the lead screw portion 270 urges the lead nut 280 and the movable base member 44 to move in a second direction parallel to the x-axis 106 (shown in FIG. 15) on the top surface 122 of the plate portion 120.

Referring to FIG. 16, the pinion gears 241, 242 are rotatably coupled to the slidable bracket 244 and are further rotatably coupled to the rack members 131, 132, respectively. Further, the pinion gears 251, 252 are rotatably coupled to the slidable bracket 254 and are further rotatably coupled to the rack members 141, 142, respectively. The slidable brackets 244, 254 are provided to move in a direction parallel to the z-axis 108 (shown in FIG. 15) utilizing the pinion gears 241, 242, 251, 252, utilizing the second movement device 52.

Second Movement Device

Referring to FIGS. 3, 14 and 15, the second movement device 52 is operably coupled to the base plate 40 and the movable base member 44. The second movement device 52 moves the movable base member 44 forward and backward parallel to the z-axis 108 (shown in FIG. 15). The second movement device 52 includes a stepper motor 302, a slidable bracket 324, a coupling member 328, a pinion gear 330, a coupling member 332, a rotatable shaft 334, a coupling member 336, a rotatable shaft 338, a coupling member 340, a pinion gear 342, and an attachment bracket 350. The stepper motor 302 has a rotatable shaft 320 that rotates in response to a control signal from the computer 90. The stepper motor 302 is supported by the slidable bracket 324. The rotatable shaft 320 is coupled to the coupling member 328 which is further coupled to the pinion gear 330. The pinion gear 330 is further coupled to the coupling member 332 which is further coupled to the rotatable shaft 334. The rotatable shaft 334 is further coupled to the coupling member 336 which is further coupled to the rotatable shaft 338. The rotatable shaft 338 is further coupled to the coupling member 340 which is further coupled to the pinion gear 342. The rotatable shaft 334 is rotatably received within the attachment bracket 350 which is attached to the movable base member 44. Further, the slidable bracket 324 is slidably disposed on the slider bar 152 of the base plate 40.

During operation, when the stepper motor 302 rotates the rotatable shaft 320 in a first rotational direction, the coupling member 328, the pinion gear 330, the coupling member 332, the rotatable shaft 334, the coupling member 336, the rotatable shaft 338, the coupling member 340, and the pinion gear 342 rotate in the first rotational direction. As a result, the pinion gears 330, 340 move on the rack members 150, 152, respectively in a first direction parallel to the z-axis 108 (shown in FIG. 15), and the attachment bracket 350 urges the movable base member 44 in the first direction parallel to the z-axis 108 (shown in FIG. 15) on the top surface 122 of the plate portion 120. Alternately, when the stepper motor 302 rotates the rotatable shaft 320 in a second rotational direction, the coupling member 328, the pinion gear 330, the coupling member 332, the rotatable shaft 334, the coupling member 336, the rotatable shaft 338, the coupling member 340, and the pinion gear 342 rotate in the second rotational direction. As a result, the pinion gears 330, 340 move on the rack members 150, 152, respectively in a second direction parallel to the z-axis 108 (shown in FIG. 15), and the attachment bracket 350 urges the movable base member 44 in the second direction parallel to the z-axis 108 (shown in FIG. 15) on the top surface 122 of the plate portion 120.

Third Movement Device

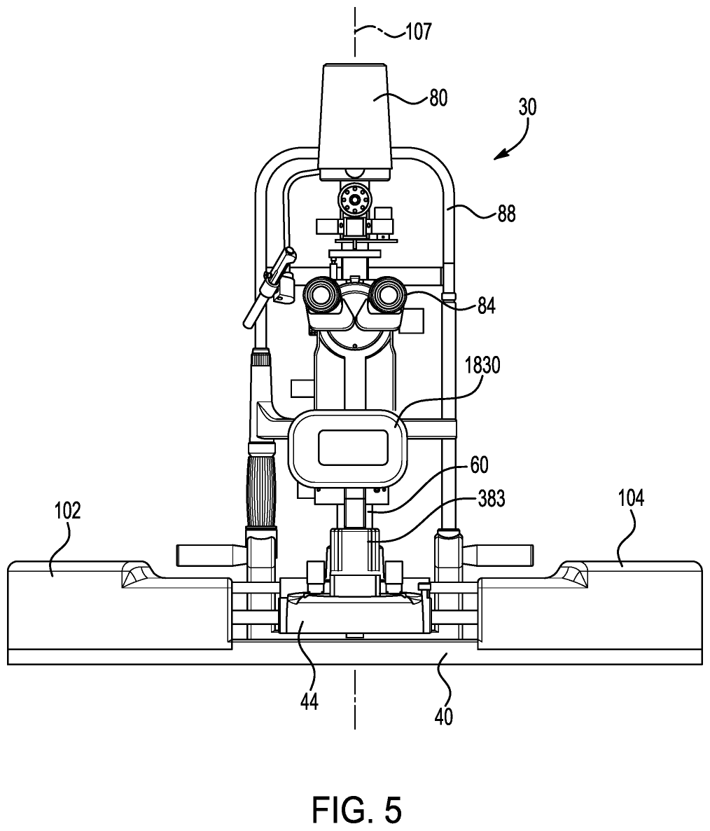

Referring to FIGS. 3, 14 and 18, the third movement device 53 is operably coupled to the movable base member 44, the lower chassis 60, and the base plate 40. The third movement device 53 moves the lower chassis 60 upwardly and downwardly parallel to the y-axis 107 (shown in FIG. 5). The third movement device 53 includes a stepper motor 383, a coupling member 390, a connecting shaft 392, a bearing fixture 394, a bearing washer 396, a C-ring 398, a bearing 400, a coupling member 410, a C-ring 412, a rotatable sleeve 420, a driving gear 422, a driven gear 430, a lead screw 432, a lead nut 434, and a bearing 438. The stepper motor 302 has a rotatable shaft 383 that rotates in response to a control signal from the computer 90. The stepper motor 383 is supported by the movable base member 44. The rotatable shaft 383 is coupled to the coupling member 390 which is further coupled to the connecting shaft 392 which extends through the aperture 190 (shown in FIG. 19) of the movable base member 44. The connecting shaft 392 is further coupled to the bearing fixture 394 which is further coupled to the bearing washer 396. The bearing washer 396 is further coupled to the bearing C-ring 398 which is further coupled to the bearing 400. The bearing 400 is further coupled to the coupling member 410 which is further coupled to the C-ring 412. The coupling member 410 is further coupled to the driving gear 420 which is further coupled to the rotatable sleeve 420. The rotatable sleeve 420 is disposed against the top surface 122 of the base plate 40 (shown in FIG. 14). The driving gear 420 is further operably coupled to the driven gear 430 which is further coupled to the lead screw 432. The lead nut 434 is disposed on the lead screw 432 and is further coupled to the bearing 438. The bearing 438 is disposed against an inner surface of the lower chassis 60.

During operation, when the stepper motor 383 rotates the rotatable shaft 386 in a first rotational direction, the coupling member 390, the connecting shaft 392, the coupling member 410, the rotatable sleeve 420, and the driving gear 422 rotate in a first rotational direction. Further, the driving gear 422 rotates the driven gear 430 in a second rotational direction (opposite to the first rotational direction). Further, the driven gear 430 rotates the lead screw 432 in the second rotational direction which induces the lead nut 434 to move upwardly and parallel to the z-axis 108 (shown in FIG. 15)—which further moves the lower chassis 60 upwardly and parallel to the z-axis 108. Alternately, when the stepper motor 383 rotates the rotatable shaft 386 in a second rotational direction, the coupling member 390, the connecting shaft 392, the coupling member 410, the rotatable sleeve 420, and the driving gear 422 rotate in the second rotational direction. Further, the driving gear 422 rotates the driven gear 430 in a first rotational direction. Further, the driven gear 430 rotates the lead screw 432 in the first rotational direction which induces the lead nut 434 to move downwardly and parallel to the z-axis 108 (shown in FIG. 15)—which further moves the lower chassis 60 downwardly and parallel to the z-axis 108.

Lower Chassis

Referring to FIGS. 14, 22 and 23, the lower chassis 60 is operably coupled to the movable base member 44. The lower chassis 60 is further coupled to the first support arm 71 and the second support arm 72 via a pin 528 (shown in FIG. 22). The lower chassis 60 includes a housing 470 having apertures 474, 476, 478 extending therethrough. The lower chassis 60 is coupled to the chassis receiving portion 178 (shown in FIG. 14) of the movable base member 44. In an exemplary embodiment, the lower chassis 60 is constructed of a metal such as steel or aluminum.

First Support Arm

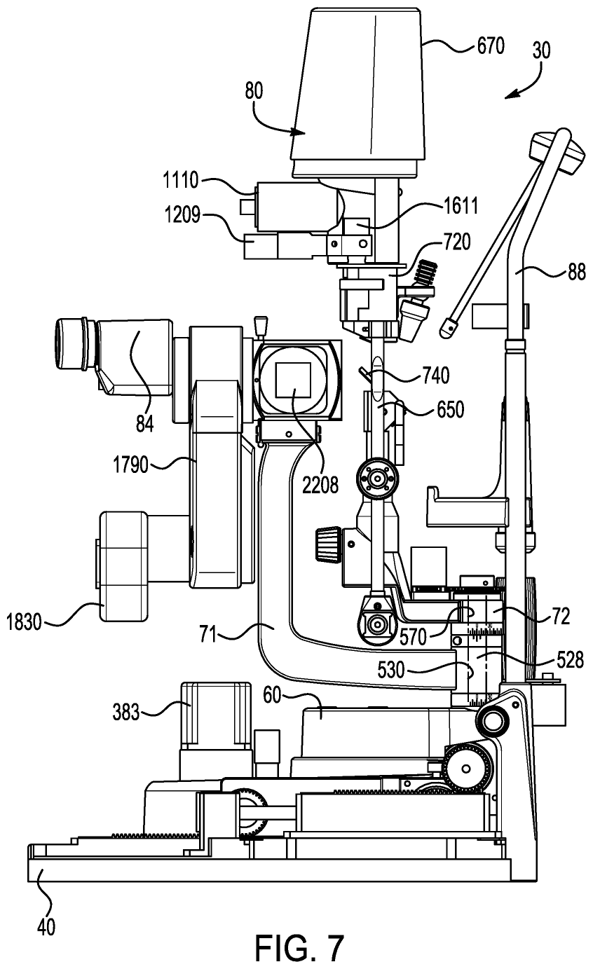

Referring to FIGS. 6, 7 and 22, the first support arm 71 is provided to support the microscope assembly 84 thereon. The first support arm 71 includes a hub portion 520, a horizontal arm portion 522, a vertical arm portion 524, a coupling member 526, and a pin 528. The hub portion 520 includes an aperture 530 (shown in FIG. 7) therethrough for receiving the pin 528 therethrough. The horizontal arm portion 522 is coupled to the hub portion 520 and extends outwardly from the hub portion 520. The vertical arm portion 524 is coupled to the horizontal arm portion 522 and extends upwardly from the horizontal arm portion 522. The coupling member 526 is coupled to a distal end of the vertical arm portion 524. The coupling member 526 is configured to hold the microscope assembly 84 thereon and is coupled to the microscope assembly 84. In an exemplary embodiment, the first support arm 71 is constructed of a metal such as steel or aluminum.

Second Support Arm

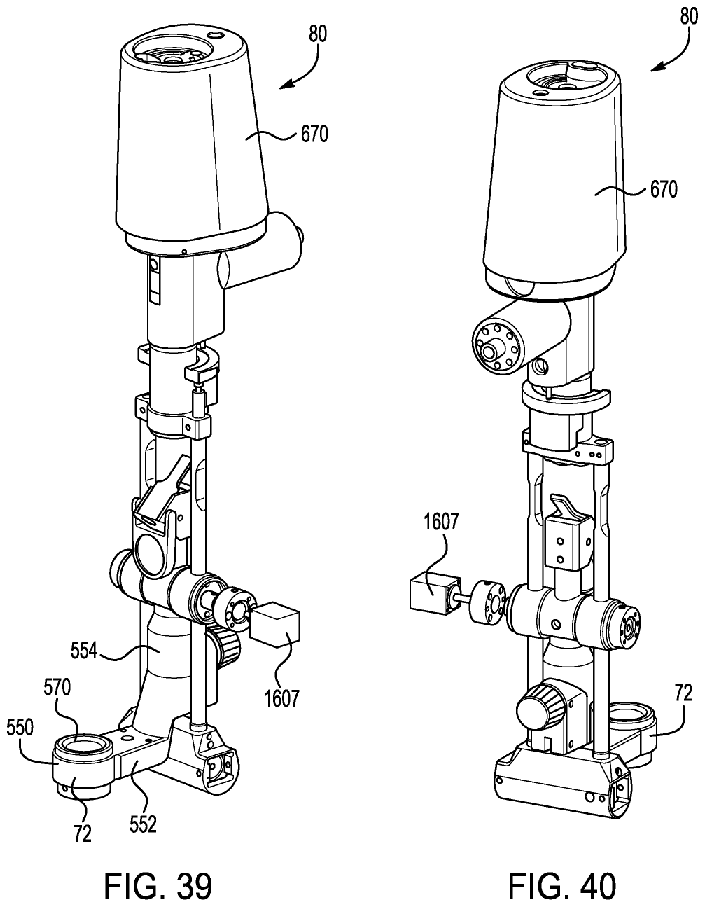

Referring to FIGS. 6, 11, 22 and 39, the second support arm 72 is provided to rotatably support the illumination assembly 80 thereon. The second support arm 72 includes a hub portion 550 (shown in FIG. 39), a horizontal arm portion 552, a vertical arm portion 554, a hub portion 556 (shown in FIG. 22), and a tip portion 557. The hub portion 550 includes an aperture 570 therethrough for receiving the pin 528 (shown in FIG. 7) therethrough. The horizontal arm portion 552 is coupled to the hub portion 550 and extends outwardly from the hub portion 550. The vertical arm portion 554 is coupled to the horizontal arm portion 552 and extends upwardly from the horizontal arm portion 552. The hub portion 556 is coupled to a distal end of the vertical arm portion 554. The tip portion 557 is coupled to the hub portion 556 and extends upwardly from the hub portion 556.

Illumination Assembly

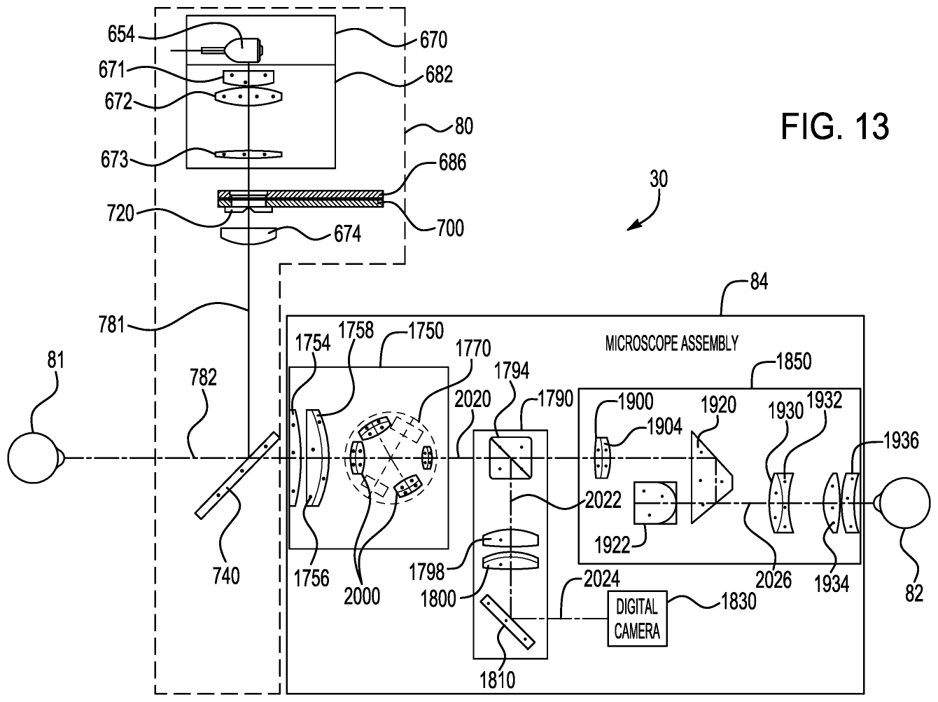

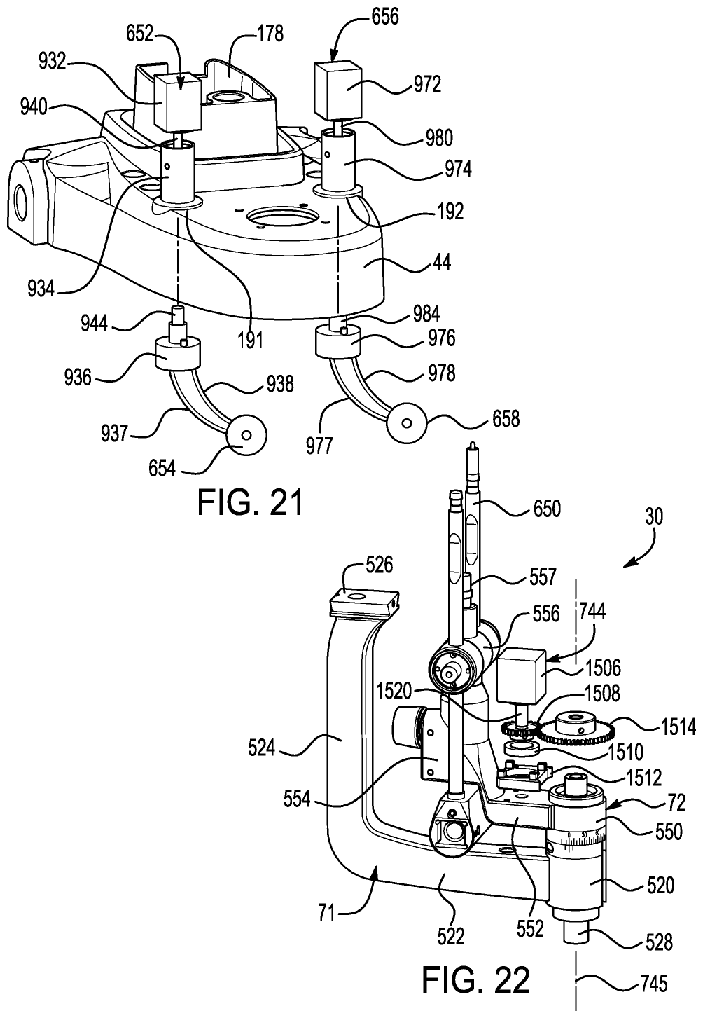

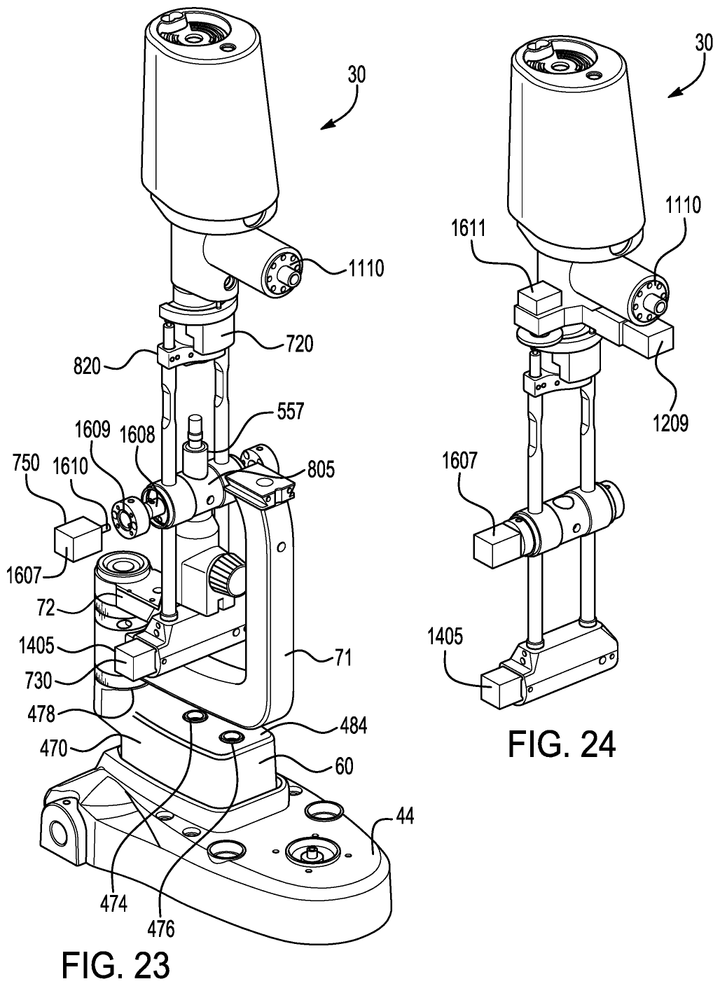

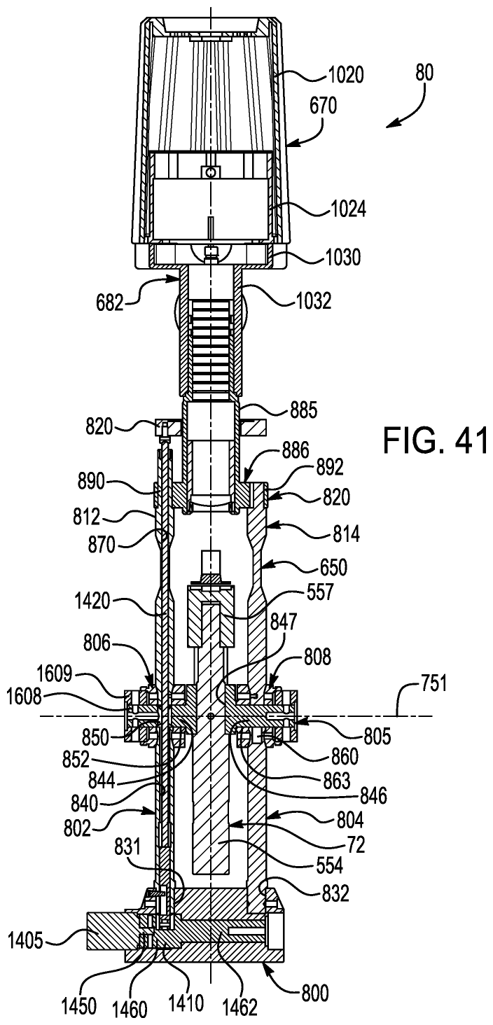

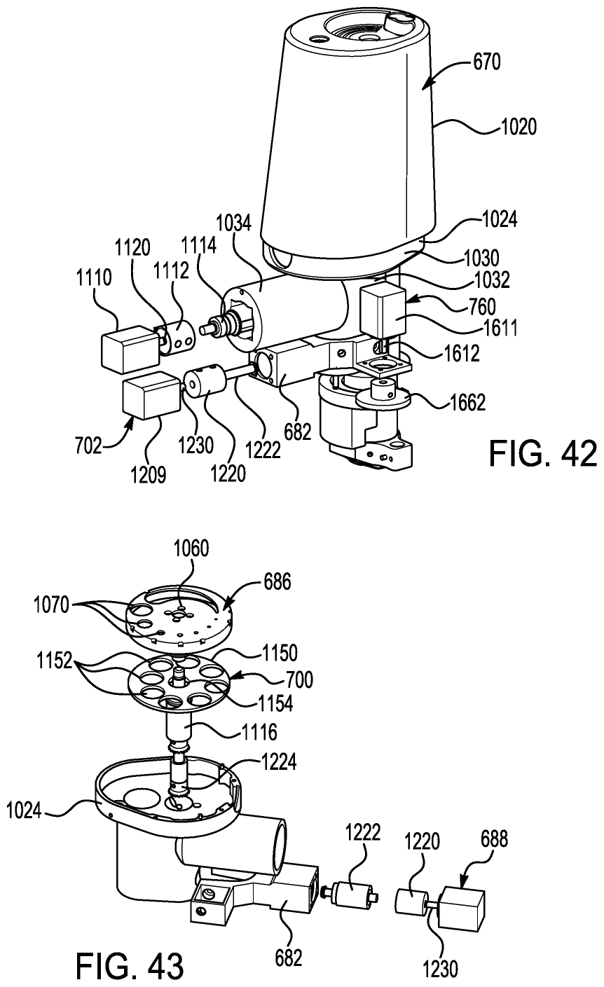

Referring to FIGS. 3, 11, 13, 32, 37, 41 and 43, the illumination assembly 80 is provided to emit light onto a subject's eye 81. The illumination assembly 80 includes a frame assembly 650, a first light intensity adjustment device 652 (shown in FIG. 21), a first light source device 654, a second light intensity adjustment device 656, a second light source device 658, a light source housing 670, condenser lens 671, 672, 673, 674 (shown in FIG. 13), a central housing 682 (shown in FIG. 41), a rotatable disk 686 (shown in FIG. 43), a light diameter adjustment device 688, a filtered glass lens assembly 700 (shown in FIG. 43), a filtered glass lens selection device 702, a slit forming device 720 (shown in FIG. 34), a slit size adjustment device 730 (shown in FIG. 23), a mirror 740 (shown in FIG. 6), a first rotation device 744 (shown in FIG. 22), a second rotation device 750 (shown in FIG. 23), a third rotation device 760 (shown in FIG. 42), and an optical axes 781, 782 (shown in FIG. 13).

Frame Assembly

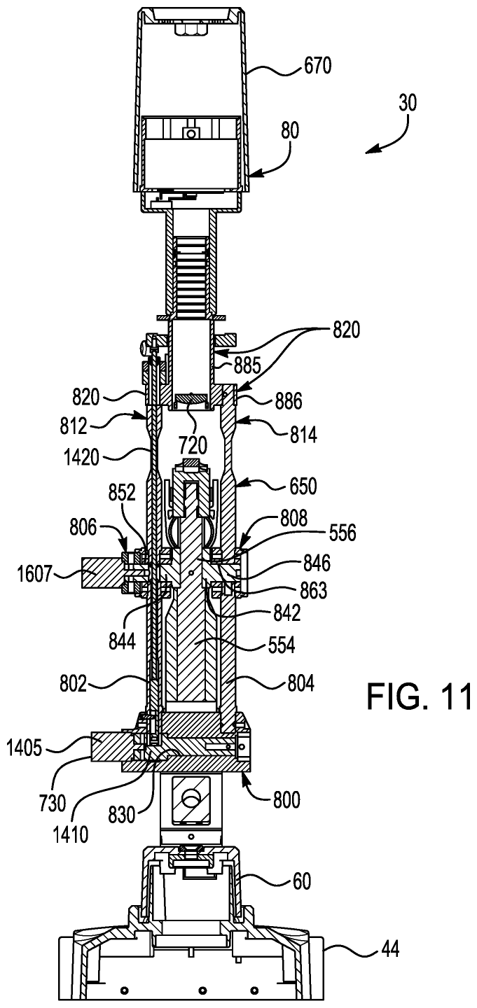

Referring to FIGS. 11, 37 and 38, the frame assembly 650 is provided to hold the remaining components of the illumination assembly 80 thereon. The frame assembly 650 includes a hub portion 800, rod portions 802, 804, a hub portion 805 (shown in FIG. 41), coupling members 806, 808, rod portions 812, 814, and the coupling member 820.

Hub Portion

Referring to FIGS. 11 and 41, the hub portion 800 is provided to hold the stepper motor 1405 thereon. The hub portion 800 includes a central cavity 830, a first aperture 831, and a second aperture 832. The central cavity 830 extends horizontally through the entire length of the hub portion 800. The first aperture 831 extends vertically into the hub portion 800 a predetermined distance. Further, the second aperture 832 extends vertically into the hub portion a predetermined distance. The central cavity 830 is provided to receive the shaft 1462 of the slit size adjustment device 730 therethrough. The first aperture 831 receives an end portion of the rod portion 802 therein for coupling the rod portion 802 to the hub portion 800. The second aperture 832 receives an end portion of the rod portion 804 therein for coupling the rod portion 804 to the hub portion 800. In an exemplary embodiment, the hub portion 800 is constructed of a metal such as steel or aluminum.

Rod Portion

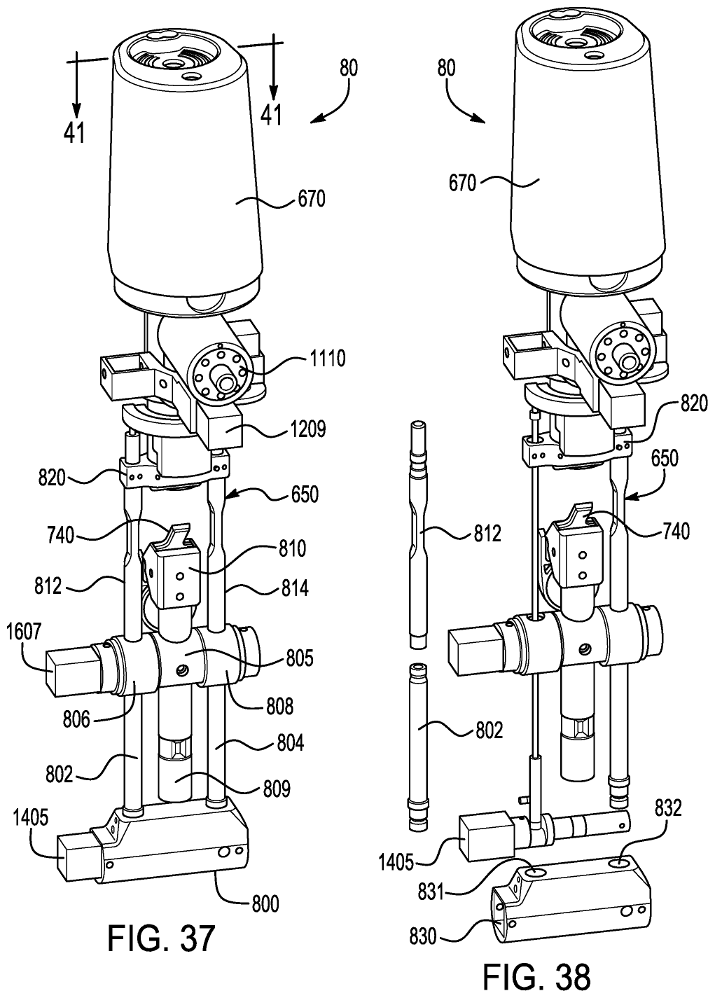

Referring to FIGS. 37, 38, and 41, the rod portion 802 is coupled to and between the hub portion 800 and the hub portion 805. The rod portion 802 includes a central aperture 840 extending longitudinally therethrough. A first end of the rod portion 802 extends into the first aperture 831 of the hub portion 800. A second end of the rod portion 802 extends into an aperture 850 of the coupling member 806. In an exemplary embodiment, the rod portion 802 is constructed of a metal such as steel or aluminum.

Rod Portion

The rod portion 804 is coupled to and between the hub portion 800 and the hub portion 805. A first end of the rod portion 804 extends into the second aperture 832 of the hub portion 800. A second end of the rod portion 804 extends into an aperture 860 of the coupling member 808. In an exemplary embodiment, the rod portion 804 is constructed of a metal such as steel or aluminum.

Hub Portion

Referring to FIGS. 11 and 41, the hub portion 805 is provided to rotatably hold the coupling members 806, 808 thereon. The hub portion 805 includes a central portion 842 (shown in FIG. 11) and pin portions 844, 846 extending outwardly from the central portion 842 in opposite directions. The central portion 842 includes an aperture 847 extending therethrough. The hub portion 805 is coupled to the second support arm 72. In particular, the vertical arm portion 554 extends through the aperture 847 of the hub portion 805. In an exemplary embodiment, the hub portion 805 is constructed of a metal such as steel or aluminum.

Coupling Member

Referring to FIGS. 11 and 41, the coupling member 806 is rotatably coupled to the pin portion 844 of the hub portion 805. The coupling member 806 includes vertical aperture 850 extending therethrough, and a horizontal aperture 852 extending therethrough. A second end of the rod portion 802 is disposed in a portion of the vertical aperture 850 to couple the rod portion 802 to the coupling member 806. A first end of the rod portion 812 is disposed in another portion of the vertical aperture 850 to couple the rod portion 812 to the coupling member 806 such that the rod portions 802, 812 are aligned with one another.

Coupling Member

The coupling member 808 is rotatably coupled to the pin portion 846 of the hub portion 808. The coupling member 808 includes vertical aperture 860 extending therethrough, and a horizontal aperture 863 extending therethrough. A second end of the rod portion 804 is disposed in a portion of the vertical aperture 860 to couple the rod portion 804 to the coupling member 808. A first end of the rod portion 814 is disposed in another portion of the vertical aperture 860 to couple the rod portion 814 to the coupling member 808 such that the rod portions 804, 814 are aligned with one another.

Rod Portion

Referring to FIG. 41, the rod portion 812 is coupled to the coupling member 806 and the coupling member 820. The rod portion 812 includes a central aperture 870 extending longitudinally therethrough that is aligned with the central aperture 840. A first end of the rod portion 812 extends into the vertical aperture 850 of the coupling member 806. A second end of the rod portion 802 extends through an aperture 890 of the coupling member 820. In an exemplary embodiment, the rod portion 812 is constructed of a metal such as steel or aluminum.

Rod Portion

The rod portion 814 is coupled to the coupling member 806 and the coupling member 820. A first end of the rod portion 814 extends into the vertical aperture 860 of the coupling member 808. A second end of the rod portion 814 extends through an aperture 892 of the coupling member 820. In an exemplary embodiment, the rod portion 814 is constructed of a metal such as steel or aluminum.

Coupling Member

Referring to FIG. 11, the coupling member 820 is coupled to the rod portions 812, 814 as discussed above. The coupling member 820 includes a tubular portion 885 and a flange portion 886. The flange portion 886 extends outwardly from the tubular portion 885 at a first end of the tubular portion 885. The flange portion 886 includes apertures 890, 892 extending therethrough. In an exemplary embodiment, the coupling member 820 is constructed of a metal such as steel or aluminum.

First Light Intensity Adjustment Device and First Light Source Device

Referring to FIGS. 3 and 21, the first light intensity adjustment device 652 is provided to adjust an amount of light emitted from the first light source device 654. The first light intensity adjustment device 652 includes a stepper motor 932, a coupling member 934, a potentiometer 936, and electrical lines 937, 938. The stepper motor 932 is coupled to the movable base member 44. The stepper motor 932 includes a rotatable shaft 940 that is coupled to the coupling member 934. The coupling member 934 extends through the aperture 191 in the movable base member 44. The coupling member 934 is further coupled to a rotatable shaft 944 of the potentiometer 936. The electrical lines 937, 938 extend from the potentiometer 936 to the first light source device 654. In an exemplary embodiment, the first light source device 654 is a light-emitting diode.

During operation, when the stepper motor 932 rotates the rotatable shaft 940 in a first rotational direction in response to a control signal from the computer 90, the coupling member 934 and the rotatable shaft 944 rotate in the first rotational direction—which induces the first light source device 654 to increase the amount of light being emitted therefrom. Alternately, when the stepper motor 932 rotates the rotatable shaft 940 in a second rotational direction in response to another control signal from the computer 90, the coupling member 934 and the rotatable shaft 944 rotate in the second rotational direction—which induces the first light source device 654 to decrease the amount of light being emitted therefrom.

Second Light Intensity Adjustment Device and Second Light Source Device

The second light intensity adjustment device 656 is provided to adjust an amount of light emitted from the second light source device 658. The second light intensity adjustment device 656 includes a stepper motor 972, a coupling member 974, a potentiometer 976, and electrical lines 977, 978. The stepper motor 972 is coupled to the movable base member 44. The stepper motor 972 includes a rotatable shaft 984 that is coupled to the coupling member 974. The coupling member 974 extends through the aperture 192 (shown in FIG. 20) in the movable base member 44. The coupling member 974 is further coupled to a rotatable shaft 984 of the potentiometer 976. The electrical lines 977, 978 extend from the potentiometer 976 to the second light source device 658. In an exemplary embodiment, the second light source device 658 is a light-emitting diode.

During operation, when the stepper motor 972 rotates the rotatable shaft 980 in a first rotational direction in response to a control signal from the computer 90, the coupling member 974 and the rotatable shaft 984 rotate in the first rotational direction—which induces the second light source device 658 to increase the amount of light being emitted therefrom. Alternately, when the stepper motor 972 rotates the rotatable shaft 980 in a second rotational direction in response to another control signal from the computer 90, the coupling member 974 and the rotatable shaft 984 rotate in the second rotational direction—which induces the second light source device 658 to decrease the amount of light being emitted therefrom.

Light Source Housing

Referring to FIGS. 21, 41 and 42, the light source housing 670 is provided to enclose the first and second light source devices 654, 658 therein. The light source housing 670 includes a cover portion 1020 and a housing portion 1024. The housing portion 1024 is coupled to the upper tubular portion 1030 of the central housing 682. The cover portion 1020 is coupled to the housing portion 1024 to define an interior region therein for holding the first and second light source devices 654, 658 therein.

Central Housing

Referring to FIGS. 41 and 42, the central housing 682 is coupled to and between the tubular portion 885 of the coupling member 820 and the housing portion 1024 of the light source housing 670. The central housing 682 includes an upper tubular portion 1030, a lower tubular portion 1032, and an extension portion 1034 (shown in FIG. 42). The upper tubular portion 1030 is coupled to the lower tubular portion 1032. Further, the extension portion 1030 extends horizontally outwardly from the lower tubular portion 1032. In particular, the upper tubular portion 1030 is coupled to the housing portion 1024. Further, the lower tubular portion 1032 receives a portion of the tubular portion 885 therein. In an exemplary embodiment, the central housing 682 is constructed of a metal such as steel or aluminum.

Referring to FIGS. 13 and 41, the central housing 682 has the condenser lens 671, 672, 673 disposed therein. The condenser lens 671, 672, 673 are aligned along an optical axis 781 such that light from the first light source device 654 passes through the condenser lens 671, 672, 673.

Rotatable Disk

Referring to FIGS. 13 and 43, the rotatable disk 686 is disposed within the housing portion 1024 of the light source housing 670. The rotatable disk 686 includes a central aperture 1060 and a plurality of apertures 1070 extending therethrough. The central aperture 1060 receives an end of a gear assembly 1116 of a light diameter adjustment device 688 therethrough. Each of the apertures of the plurality of apertures 1070 has a different diameter, which is used to determine a diameter of a light beam passing through the respective aperture.

Light Diameter Adjustment Device

Referring to FIGS. 42 and 43, the light diameter adjustment device 688 is operably coupled to the rotatable disk 686 and is provided to rotate the rotatable disk 686 such that one aperture of the plurality of apertures 1070 is aligned with an optical axis 781 (shown in FIG. 13) such that light from the first light source device 654 passes through the aperture thereof. The light diameter adjustment device 688 includes a stepper motor 1110, a coupling member 1112, a gear assembly 1114, and a gear assembly 1116. The stepper motor 1110 includes a rotatable shaft 1120 that is coupled to the coupling member 1112. The coupling member 1112 is further coupled to the gear assembly 1114. The gear assembly 1114 (shown in FIG. 42) is disposed within the extension portion 1034 of the central housing 682. The gear assembly 1114 is further rotatably coupled to the gear assembly 1116 (shown in FIG. 43) which is coupled to the rotatable disk 686.

During operation, when the stepper motor 1110 rotates the rotatable shaft 1120 in a first rotational direction in response to a control signal from the computer 90, the coupling member 1112 and the gear assembly 1114 rotate in the first rotational direction—which induces the gear assembly 1116 and the rotatable disk 686 to rotate in a second rotational direction a predetermined distance—to align an aperture of the plurality of apertures 1070 in the optical axis 781 (shown in FIG. 13). As a result, the aperture receives light which is passed through the condenser lenses 671, 672, 673 from the first light source device 654.

Filtered Glass Lens Assembly

The filtered glass lens assembly 700 is disposed within the housing portion 1024 of the light source housing 670 underneath the rotatable disk 686. The filtered glass lens assembly 700 includes a rotatable disk 1150 having a central aperture 1154. The rotatable disk 1150 further includes a plurality of filtered glass lenses 1152 each disposed in respective holes extending through the rotatable disk 1150. The central aperture 1154 receives a portion of the gear assembly 1224 therethrough. The filtered glass lens assembly 700 is coupled to a portion of the gear assembly 1224. Further, each lens of the plurality of filtered glass lens 1152 has a distinct color.

Filtered Glass Lens Selection Device

The filtered glass lens selection device 702 is operably coupled to the filtered glass lens assembly 700 and is provided to rotate the assembly 700 such that one lens of the plurality of filtered glass lens 1152 is aligned with an optical axis 781 (shown in FIG. 13) such that light from the rotatable disk 686 passes through the lens of the assembly 700. The filtered glass lens selection device 702 includes a stepper motor 1209, a coupling member 1220, a gear assembly 1222, and a gear assembly 1224. The stepper motor 1209 includes a rotatable shaft 1230 that is coupled to the coupling member 1220. The coupling member 1220 is further coupled to the gear assembly 1222. The gear assembly 1222 is disposed within the central housing 682. The gear assembly 1222 is further rotatably coupled to the gear assembly 1224 which is coupled to the rotatable disk 1150 of the filtered glass lens assembly 700.

During operation, when the stepper motor 1209 rotates the rotatable shaft 1230 in a first rotational direction in response to a control signal from the computer 90, the coupling member 1220 and the gear assembly 1222 rotate in the first rotational direction—which induces the gear assembly 1224 and the rotatable disk 1150 of the assembly 700 to rotate in a second rotational direction a predetermined distance—to align a filtered glass lens of the plurality of filtered glass lenses 1152 in the optical axis 781 (shown in FIG. 13). As a result, the filtered glass lens receives light which is passed through the rotatable disk 686 from the first light source device 654.

Slit Forming Device

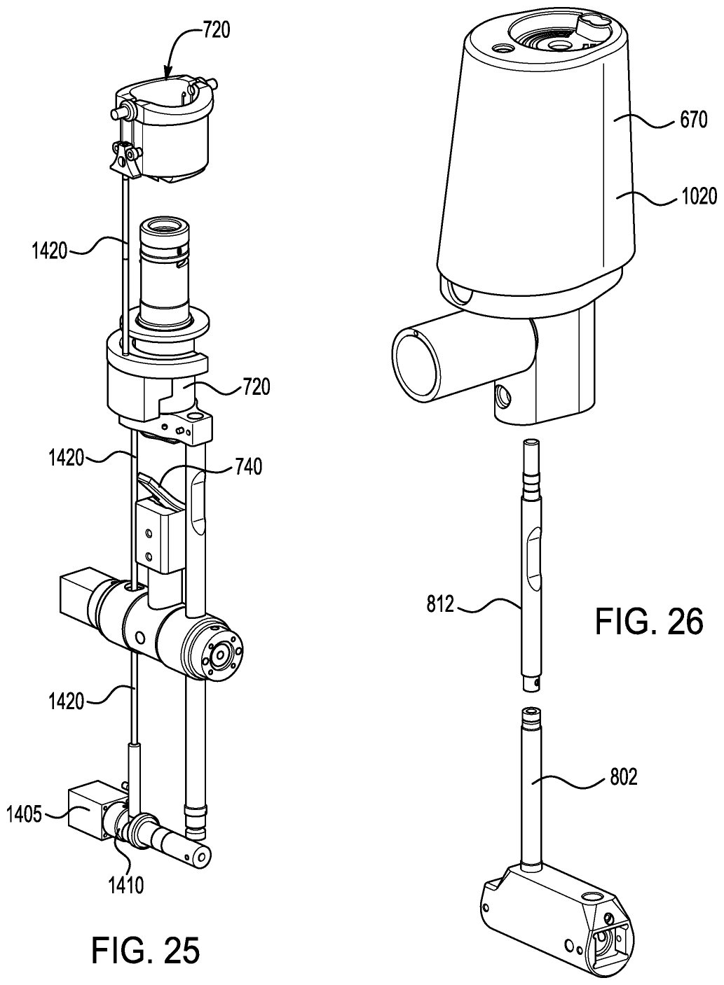

Referring to FIGS. 25-36, the slit forming device 720 is provided to form a slit for receiving light therethrough that is output from the light source device 754. The slit forming device 720 is disposed within the tubular portion 885 of the coupling member 820 (shown in FIG. 11), and underneath the filtered glass lens assembly 700.

Referring to FIGS. 34-36, the slit forming device 720 includes a first housing portion 1301, a second housing portion 1302, pivot pins 1310, 1312, a first side pin 1321, a second side pin 1322, a first blade 1331, a second blade 1332, a first spring 1341 (shown in FIG. 32), and a second spring 1342.

The first and second housing portions 1301, 1302 are pivotally attached to one another utilizing the pivot pins 1310, 1312. The pivot pin 1310 extends through a first side of the first housing portion 1301, and a first side of the second housing portion 1302. Further, the pivot pin 1312 extends through a second side of the second housing portion 1302, and a second side of the second housing portion 1302.

The first side pin 1321 is coupled to the first side of the first housing portion 1301 below the pivot pin 1310. Further, the second side pin 1322 is coupled to the first side of the second housing portion 1302 below the pivot pin 1310. The first and second side pins 1321, 1322 are contacted by a wedge-shaped member 1430 of the slit size adjustment device 730 (shown in FIG. 11) for opening a gap between the bottom end of the first and second housing portions 1301, 1302.

The first blade 1331 is coupled to a bottom surface of the first housing portion 1301. The second blade 1332 is coupled to a bottom surface of the second housing portion 1302. A gap between the first and second blade portions 1331, 1332 forms a slit for allowing light to pass therethrough.

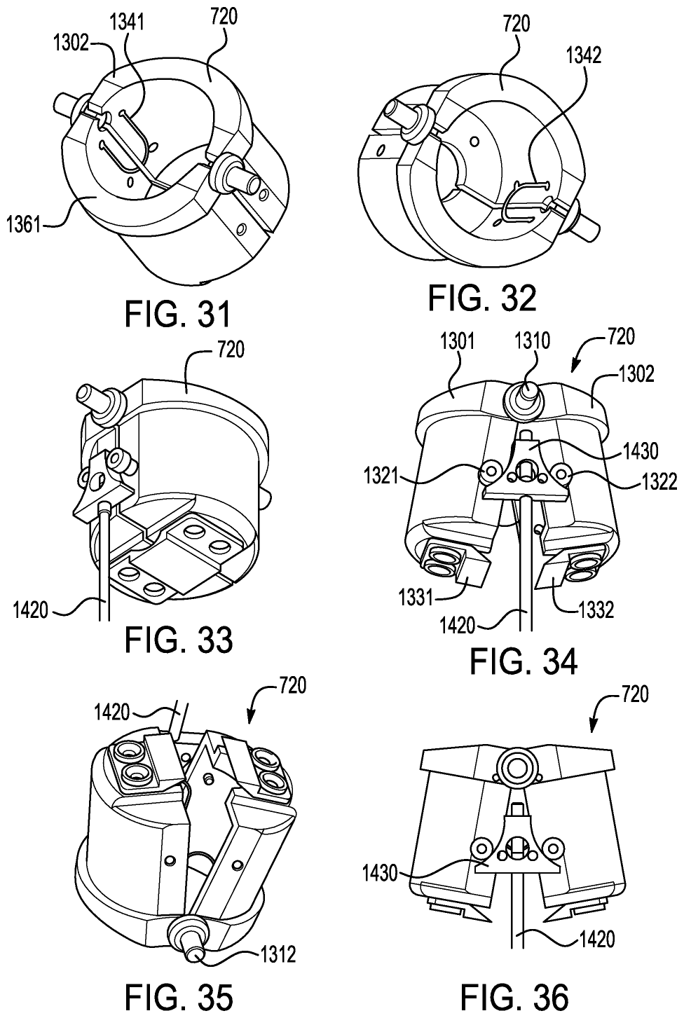

Referring to FIGS. 31 and 32, the first spring 1341 is coupled to both the first and second housing portions 1301, 1302 and is provided to urge the housing portions 1301, 1302 in a closed position such that the first and second blades 1331, 1332 contact one another. Further, the second spring 1342 is coupled to both the first and second housing portions 1301, 1302 is provided to urge the housing portions 1301, 1302 in a closed position such that the first and second blades 1331, 1332 contact one another.

In an exemplary embodiment, the first and second housing portions 1301, 1302, the pivot pins 1310, 1312, the first and second side pins 1321, 1322, and the first and second blades 1331, 1332 are constructed of a metal such as steel or aluminum. Further, the first and second springs 1341, 1342 are constructed of a metal such as steel.

Slit Size Adjustment Device

Referring to FIGS. 11, 25, 34 and 41, the slit size adjustment device 730 is operably coupled to the slit forming device 720 and is provided to move the first and second housing portions 1301, 1302 and the first and second blades 1331, 1332 to obtain a desired slit size therebetween. The slit size adjustment device 730 includes a stepper motor 1405, a cam member 1410 (shown in FIG. 41), push rod 1420, and a wedge-shaped member 1430 (shown in FIG. 34).

Referring to FIGS. 11 and 41, the stepper motor 1405 includes a rotatable shaft 1450 which is coupled to a cam member 1410. The cam member 1410 includes a cam portion 1460 and a shaft 1462. The shaft 1462 is coupled to the cam portion 1460 and extends horizontally away from the cam portion 1460. The cam member 1410 is disposed within an aperture 830 (shown in FIG. 11) of the hub member 800.

The push rod 1420 is operably coupled to the cam member 1410. Further, the pushrod 1420 extends through the central aperture 840 (shown in FIG. 41) of the rod portion 802 and the central aperture 870 of the rod portion 812. An end of the pushrod 1420 is coupled to the wedge-shaped member 1430 (shown in FIG. 43).

During operation, when the stepper motor 1405 rotates the rotatable shaft 1450 in a first rotational direction in response to a control signal from the computer 90, the cam member 1410 rotates in the first rotational direction. Further, the rotating cam portion 1460 of the cam member 1410 moves the push rod 1420 and the wedge-shaped member 1430 upwardly such that the first and second blades 1331, 1332 of the slit forming device 720 are moved apart from one another—to increase a slit size therebetween. Alternately, when the stepper motor 1405 rotates the rotatable shaft 1450 in a second rotational direction in response to another control signal from the computer 90, the cam member 1410 rotates in a second rotational direction. Further, the rotating cam portion 1460 of the cam member 1410 moves the push rod 1420 and the wedge-shaped member 1430 downwardly such that the first and second blades 1331, 1332 of the slit forming device 720 are moved toward one another—to decrease the slit size therebetween.

First Rotation Device

Referring to FIGS. 3, 7 and 22, the first rotation device 744 is provided to rotate the illumination assembly 80 about a vertical axis 745 that extends through the second support arm 72 and the lower chassis 60. The first rotation device 744 includes a stepper motor 1506, a gear member 1508, a bearing 1510, a mounting bracket 1512, and a gear member 1514. The stepper motor 1506 includes a rotatable shaft 1520 is coupled to the gear member 1508. The gear member 1508 threadably engages the gear member 1514. The gear member 1514 is coupled to the stationary pin 528. The gear member 1508 is supported by the bearing 1510 which is disposed on the mounting bracket 1512. The mounting bracket 1512 is mounted on the second support arm 72 and is utilized to hold the stepper motor 1506 on the second support arm 72.

During operation, when the stepper motor 1506 rotates the rotatable shaft 1520 in a first rotational direction in response to a control signal from the computer 90, the gear member 1508 rotates in the first rotational direction. Further, since the gear member 1514 and the pin 528 are stationary, the stepper motor 1506 and the illumination assembly 80 rotate in the first rotational direction about the vertical axis 745. Alternately, when the stepper motor 1506 rotates the rotatable shaft 1520 in a second rotational direction in response to another control signal from the computer 90, the gear member 1508 rotates in the second rotational direction. Further, since the gear member 1514 and the pin 528 are stationary, the stepper motor 1506 and the illumination assembly 80 rotate in the second rotational direction about the vertical axis 745.

Second Rotation Device

Referring to FIGS. 3, 23 and 41, the second rotation device 750 is provided to rotate the illumination assembly 80 about a horizontal axis 751 (shown in FIG. 41) extending through the hub portion 805. The second rotation device 750 includes a stepper motor 1607 and a coupling member 1609. The stepper motor 1607 includes a rotatable shaft 1610 that is coupled to a shaft member 1608 that is further coupled to the coupling member 806 (shown in FIG. 41). The coupling member 806 is rotatable on the stationary pin portion 844 about the horizontal axis 751 (shown in FIG. 41). Further, the housing of the stepper motor 607 is coupled to the coupling member 609 which is further coupled to the stationary pin portion 844.

During operation, when the stepper motor 1607 rotates the rotatable shaft 1610 in a first rotational direction in response to a control signal from the computer 90, the coupling members 806, 808 rotate in the first rotational direction on the pin portions 844, 846, respectively, about the horizontal axis 751—such that the illumination assembly 80 rotates in the first rotational direction. Alternately, when the stepper motor 1607 rotates the rotatable shaft 1610 in a second rotational direction in response to another control signal from the computer 90, the coupling members 806, 808 rotate in the second rotational direction on the pin portions 844, 846, respectively, about the horizontal axis 751—such that the illumination assembly 80 rotates in the second rotational direction.

Third Rotation Device

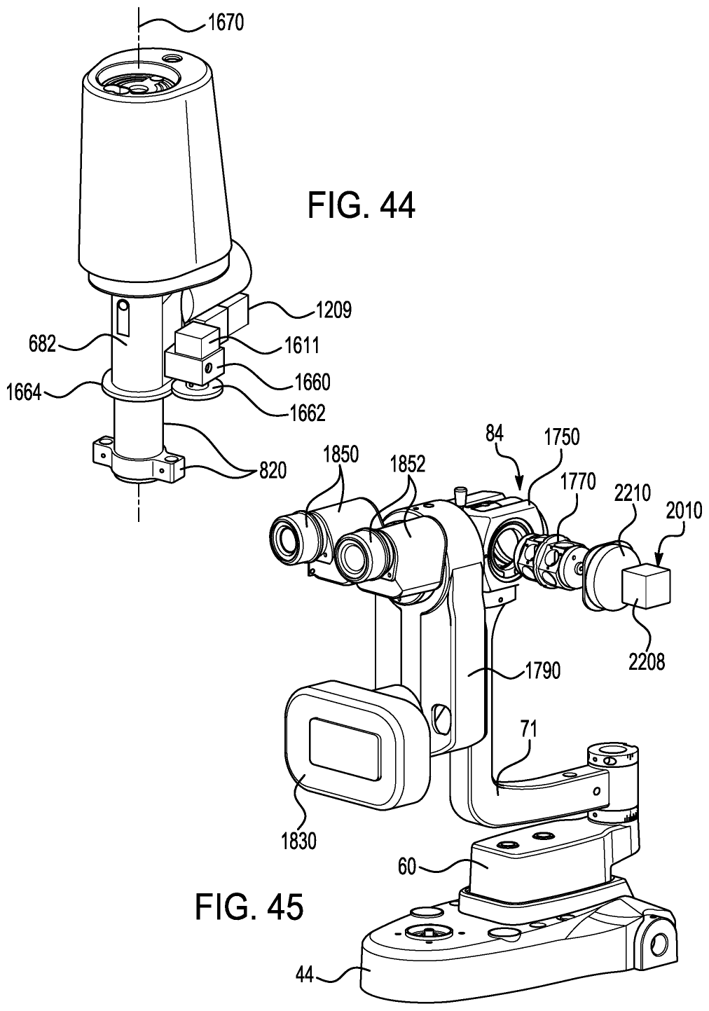

Referring to FIGS. 3, 42 and 44, the third rotation device 760 is provided to rotate the slit forming device 720 and the light source housing 670 (holding the first and second light source devices 654, 658 therein) about a vertical axis 1670 (shown in FIG. 44). The third rotation device 760 includes a stepper motor 1611, a mounting bracket 1660, a gear member 1662, and a gear member 1664. The stepper motor 1611 includes a rotatable shaft 1612 that is coupled to a gear member 1662. The stepper motor 1611 is mounted on a mounting bracket 1660 which is further mounted on the central housing 682. The gear member 1662 is threadably coupled to a stationary gear member 1664. The stationary gear member 1664 is mounted to the central housing 682.

During operation, when the stepper motor 1611 rotates the rotatable shaft 1612 in a first rotational direction in response to a control signal from the computer 90, the gear member 1662 rotates in the first rotational direction on the gear member 1664—such that the slit forming device 720 and the light source housing 670 rotate in the first rotational direction about the vertical axis 1670. Alternately, when the stepper motor 1611 rotates the rotatable shaft 1612 in a second rotational direction in response to another control signal from the computer 90, the gear member 1662 rotates in the second rotational direction on the gear member 1664—such that the slit forming device 720 and the light source housing 670 rotate in the second rotational direction about the vertical axis 1670.

Microscope Assembly

Referring to FIGS. 7, 13, 45 and 46, the microscope assembly 84 is provided to receive light from the subject's eye 81 (shown in FIG. 13), and allow a magnified image of the subjects eye 81 to be viewed through eye piece housings 1850, 1852 by a viewer's eye 82 (shown in FIG. 13). Further, the microscope assembly 84 is provided to generate a digital image from the light received from the subject's eye 81 in response to a control signal from the computer 90, and to send the digital image to the computer 90.

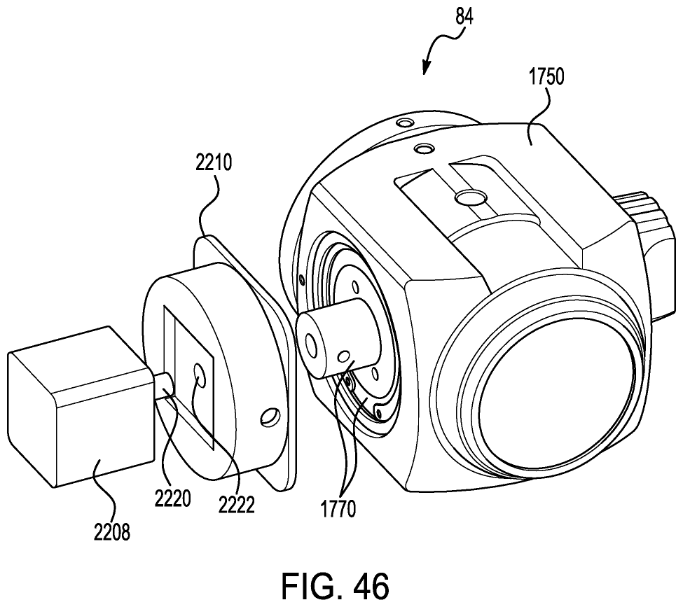

The microscope assembly 84 includes a lens housing portion 1750 (shown in FIG. 45), condenser lens 1754, 1756, 1758 (shown in FIG. 13), a magnification lens assembly 1770, a vertical housing portion 1790 (shown in FIG. 7), a beam splitter 1794 (shown in FIG. 13), condenser lens 1798, 1800, a mirror 1810, a digital camera 1830, eyepiece housings 1850, 1852, condenser lens 1900, 1904, a prism 1920, a prism 1922, eyepiece lenses 1930, 1932, 1934, 1936, a magnification adjustment device 2010 (shown in FIG. 41), and optical axes 2020, 2022, 2024, 2026.

Lens Housing Assembly

Referring to FIGS. 13, 45 and 46, the lens housing portion 1750 is provided to hold the condenser lenses 1754, 1756, 1758 and the magnification lens assembly 1770 therein. The lens housing portion 1750 is coupled to the first support arm 71. The magnification lens assembly includes a plurality of magnification lenses 2000. The condenser lenses 1754, 1756, 1758 and the magnification lens assembly 1770 are aligned along the optical axis 2020 and receive light from the subjects eye 81 along the optical axis 2020.

Vertical Housing Portion

The vertical housing portion 1790 is provided to hold the beam splitter 1794, the condenser lenses 1798, 1800 and the mirror 1810 therein. The vertical housing portion 1790 is coupled to the lens housing portion 1750. The beam splitter 1794 is aligned along the optical axes 2020 and 2022. Further, the condenser lenses 1798, 1800 and the mirror 1810 are aligned along the optical axis 2022.

Digital Camera

Referring to FIGS. 3 and 13, the digital camera 1830 is aligned along the optical axis 2024 and receives light from the mirror 1810. Further, the digital camera 1830 generates a digital image of the subject's eye 81 using the light from the mirror 1810 in response to a control signal from the computer 90. Further, the digital camera 1830 sends the digital image to the computer 90.

Eyepiece Housings

Referring to FIGS. 13 and 45, the eyepiece housings 1850, 1852 have optical components therein that allow a user to view an image of the subject's eye 81. The optical components disposed in each of the eyepiece housings 1850, 1852 have an identical structure to one another. Accordingly, only the optical components within the eyepiece housing 1850 will be described herein for purposes of simplicity. In particular, the eyepiece housing 1850 has condenser lenses 1900, 1904, a prism 1920, a prism 1922, eyepiece lenses 1930, 1932, 1934, 1936 therein. The condenser lenses 1900, 1904, and the prism 1920 are aligned along the optical axis 2020. Further, the prism 1920, the prism 1922, the eyepiece lenses 1930, 1932, 1934, 1936 are aligned along the optical axis 2026.

Magnification Adjustment Device

Referring to FIGS. 3, 13, 45 and 46, the magnification adjustment device 2010 is provided to move a magnification lens of the plurality of magnification lenses 2000 (shown in FIG. 13) into the optical axis 2020. The magnification adjustment device 2010 includes a stepper motor 2208 and an attachment member 2210. The stepper motor 2208 includes a rotatable shaft 2220 that extends through an aperture 2222 of the attachment number 2210 and is coupled to the magnification lens assembly 1770.

During operation, when the stepper motor 2208 rotates the rotatable shaft 2220 in a first rotational direction in response to a control signal from the computer 90, a lens of the plurality of magnification lenses 2000 is rotated in the first rotational direction and the lens is aligned with the optical axis 2020 to modify a magnification of received light passing through the lens.

Light Paths

Referring to FIG. 13, a brief explanation of the light paths in the illumination assembly 80 and the microscope assembly 84 will be explained. Initially, the first light source device 654 emits light along the optical axis 781 and the light passes through the condenser lenses 671, 672, 673, an aperture in the rotatable disc 686, a filter glass lens in the filtered glass lens assembly 700, a slit in the slip forming device 720, the condenser lens 674, and onto the mirror 740. The mirror 740 reflects the light along the optical axis 782 which contacts the subject's eye 81 and is reflected back from the subject's eye 81 along the optical axis 782 and passes through the condenser lenses 1754, 1756, 1758, a magnification lens in the magnification lens assembly 1770, and into the beam splitter 1794. The beam splitter 1794 splits the received light onto the optical axis 2020 and the optical axis 2022. The light from the beam splitter 1794 moving along the optical axis 2022 passes through the condenser lens 1798, 1800 to the mirror 1810. The mirror 1810 reflects the light along the optical axis 2024 to the digital camera 1830. Referring again to the beam splitter 1794, the light from the beam splitter 1794 along the optical axis 2020 passes through the condenser lenses 1900, 1904 and onto the prism 1920. The prism 1920 reflects the light onto the prism 1922 which then reflects the light along the optical axis 2026 to the eyepiece lenses 1930, 1932, 1934, 1936 and onto the viewer's eye 82.

Chin Rest Assembly

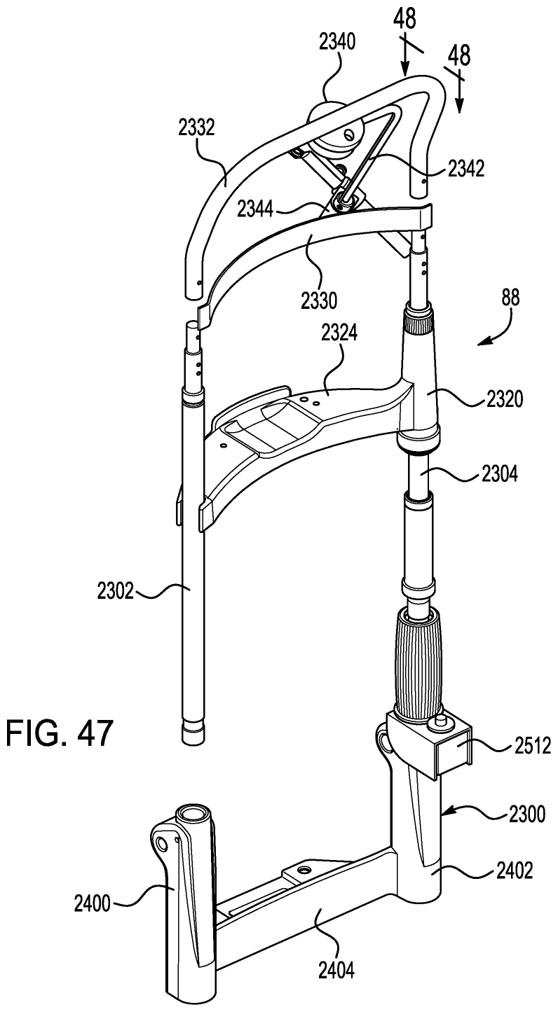

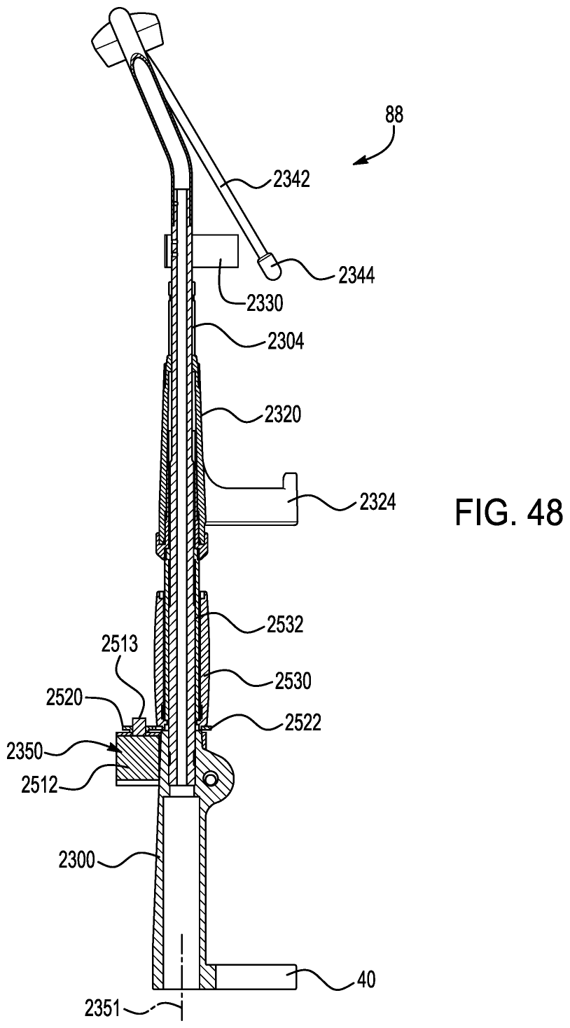

Referring to FIGS. 47 and 48, the chin rest assembly 88 is provided to support a subject's chin such that light from the illumination assembly 80 is reflected onto the subject's eye 81 (shown in FIG. 13). The chin rest assembly 88 includes a chin rest base member 2300, a central support tube 2302, a central support tube 2304, a chin rest support tube 2320, a chin rest 2324, a forehead support bar 2330, an upper support tube 2332, a rotatable holding member 2340, a light support bar 2342, a light source member 2344, and a chin rest adjustment device 2350.

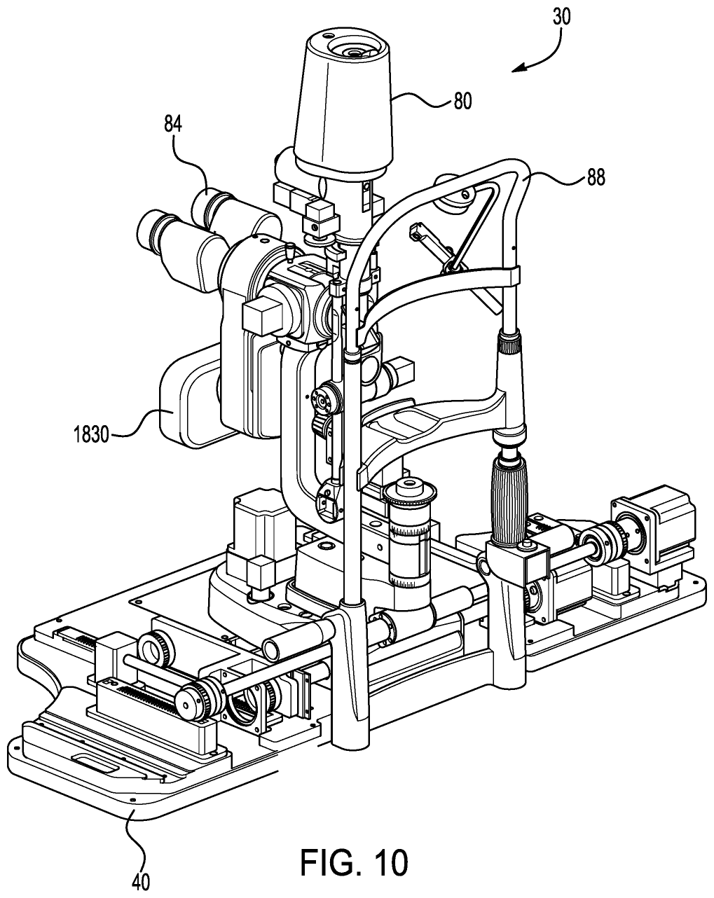

Referring to FIGS. 10, 47 and 48, the chin rest base member 2300 is provided to support the remaining components of the chin rest assembly 88. The chin rest base member 2300 is coupled to the base plate 40. The chin rest base member 2300 includes vertical tubular portions 2400, 2402 and a cross-member 2404. The vertical tubular portions 2400, 2402 are coupled to first and second ends, respectively of the cross-member 2404 and extend upwardly from the cross-member 2404. A first end of the central support tube 2302 is disposed in the vertical tubular portion 2400. Further, a first end of the central support tube 2304 is disposed in the vertical tubular portion 2402.

The chin rest 2324 is slidably disposed on and between the central support tube 2302 and the central support tube 2304. The upper support tube 2332 is substantially U-shaped and is coupled to a second end of the central support tube 2302 and a second end of the central support tube 2304. The forehead support bar 2330 is coupled to and extends across the upper support tube 2332. The rotatable holding member 2340 is coupled to the upper support tube 2332 and holds a light support bar 2342 thereon. The light support bar 2342 has a light source member 2344 coupled to an end of the light support bar 2342.

Chin Rest Adjustment Device

The chin rest adjustment device 2350 is provided to move the chin rest 2324 to a desired location along the vertical axis 2351. The chin rest adjustment device 2350 includes a stepper motor 2512, a gear member 2520, a gear member 2522, a rotatable nut 2530, and a threaded tube 2532.

The stepper motor 2512 is coupled to an exterior surface of the vertical tubular portion 2402. The stepper motor 2512 has a rotatable shaft 2513 that is coupled to a gear member 2520. The gear member 2520 is threadably coupled to a gear member 2522 that is further coupled to the rotatable nut 2530. The rotatable nut 2530 is disposed around and threadably contacts a threaded tube 2532. The threaded tube 2532 is disposed around the central support tube 2304, and the threaded tube 2532 has an upper end that is disposed against the chin rest support tube 2320.

During operation, when the stepper motor 2512 rotates the rotatable shaft 2513 in a first rotational direction in response to a control signal from the computer 90, the gear member 2520 rotates in the first operational direction, and the gear member 2522 rotates in a second rotational direction. Further, the rotatable nut 2530 rotates in the second operational direction which induces the chin rest support tube 2320 to move upwardly along the vertical axis 2351. Alternately, when the stepper motor 2512 rotates the rotatable shaft 2513 in a second rotational direction in response to a control signal from the computer 90, the gear member 2520 rotates in the second operational direction, and the gear member 2522 rotates in the first rotational direction. Further, the rotatable nut 2530 rotates in the first operational direction which induces the chin rest support tube 2320 to move downwardly along the vertical axis 2351.

Computer

Referring to FIGS. 1-9, the computer 90 is provided to control operation of the first, second, third movement devices 51, 52, 53, the illumination assembly 80, the microscope assembly 84, and the chin rest assembly 88 in response to commands received by the remote communication module 100. Referring to FIG. 3, the computer 90 includes a microprocessor 2600 operably coupled to a memory device 2602.

The computer 90 is electrically coupled to the stepper motor 201 of the first movement device 51 utilizing the electrical lines 2610, 2612. The computer 90 controls operation of the stepper motor 201 in response to commands received from the remote communication module 100.

Further, the computer 90 is electrically coupled to the stepper motor 302 in the second movement device 52 utilizing the electrical lines 2614, 2616. The computer 90 controls operation of the stepper motor 302 in response to commands received from the remote communication module 100.

Further, the computer 90 is electrically coupled to the stepper motor 383 of the third movement device 53 utilizing the electrical lines 2620, 2624. The computer 90 controls operation of the stepper motor 383 in response to commands received from the remote communication module 100.

Further, the computer 90 is electrically coupled to the stepper motor 932 of the first light intensity adjustment device 652 utilizing the electrical lines 2628, 2632. The computer 90 controls operation of the stepper motor 932 in response to commands received from the remote communication module 100.

Further, the computer 90 is electrically coupled to the stepper motor 972 of the second light intensity adjustment device 656 utilizing the electrical lines 2636, 2640. The computer 90 controls operation of the stepper motor 972 in response to commands received from the remote communication module 100.

Further, the computer 90 is electrically coupled to the stepper motor 1405 of the slit size adjustment device 730 utilizing the electrical lines 2644, 2648. The computer 90 controls operation of the stepper motor 1405 in response to commands received from the remote communication module 100.

Further, the computer 90 is electrically coupled to the stepper motor 1506 of the first rotation device 744 utilizing the electrical lines 2652, 2656. The computer 90 controls operation of the stepper motor 1506 in response to commands received from the remote communication module 100.

Further, the computer 90 is electrically coupled to the stepper motor 1607 of the second rotation device 750 utilizing the electrical lines 2660, 2664. The computer 90 controls operation of the stepper motor 1607 in response to commands received from the remote communication module 100.

Further, the computer 90 is electrically coupled to the stepper motor 2208 of the magnification adjustment device 2010 utilizing the electrical lines 2668, 2672. The computer 90 controls operation of the stepper motor 2208 in response to commands received from the remote communication module 100.

Further, the computer 90 is electrically coupled to the stepper motor 1209 of the filtered glass lens selection device 702 utilizing the electrical lines 2676, 2680. The computer 90 controls operation of the stepper motor 1209 in response to commands received from the remote communication module 100.

Further, the computer 90 is electrically coupled to the stepper motor 1110 of the light diameter adjustment device 688 utilizing the electrical lines 2684, 2688. The computer 90 controls operation of the stepper motor 1110 in response to commands received from the remote communication module 100.

Further, the computer 90 is electrically coupled to the stepper motor 1611 of the third rotation device 760 utilizing the electrical lines 2692, 2696. The computer 90 controls operation of the stepper motor 1611 in response to commands received from the remote communication module 100.

Further, the computer 90 is electrically coupled to the stepper motor 2512 of the chin rest adjustment device 2350 utilizing the electrical lines 2700, 2704. The computer 90 controls operation of the stepper motor 2512 in response to commands received from the remote communication module 100.

Further, the computer 90 operably communicates with the digital camera 1830 in the microscope assembly 84 utilizing the communication bus 2800. The computer 90 controls operation of the digital camera 1830 in response to commands received from the remote communication module 100.

Further, the computer 90 operably communicates with the remote communication module 100 utilizing the communication bus 2810.

Remote Communication Module

The remote communication module 100 is provided to receive remote control messages from a remote computer 32 utilizing the communication network 35. The remote control messages contain commands for controlling operation of the first movement device 51, the second movement device 52, the third movement device 53, the first light intensity adjustment device 652, the second light intensity adjustment device 656, the slit size adjustment device 730, the first rotation device 744, the second rotation device 750, the magnification adjustment device 2010, the filtered glass lens selection device 702, the light diameter adjustment device 688, the third rotation device 760, the chin rest adjustment device 2350, and the microscope assembly 84.

Flowchart

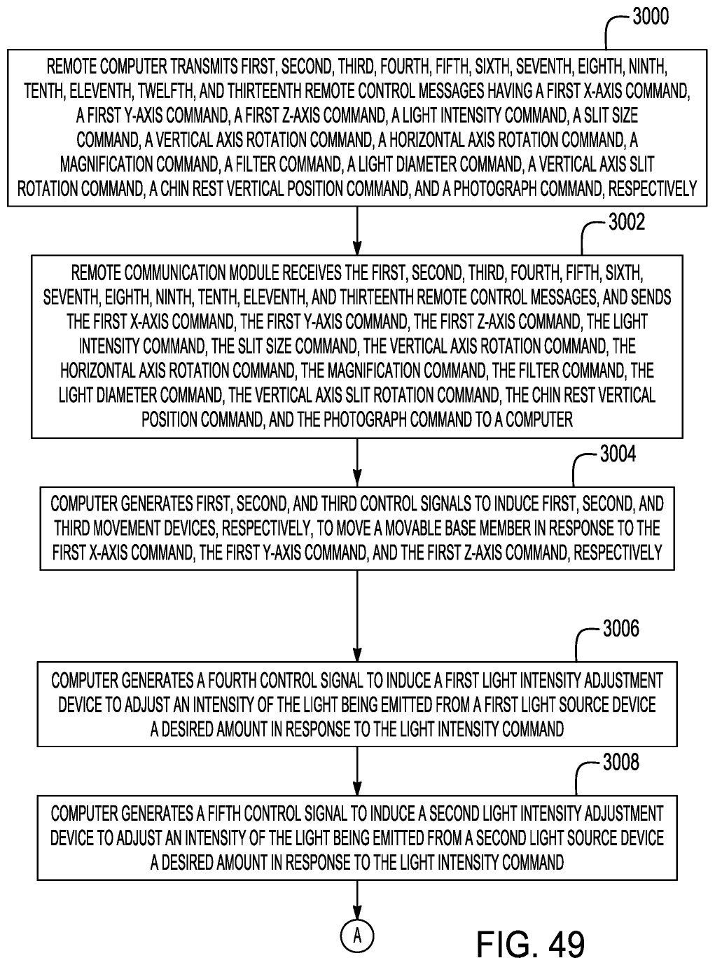

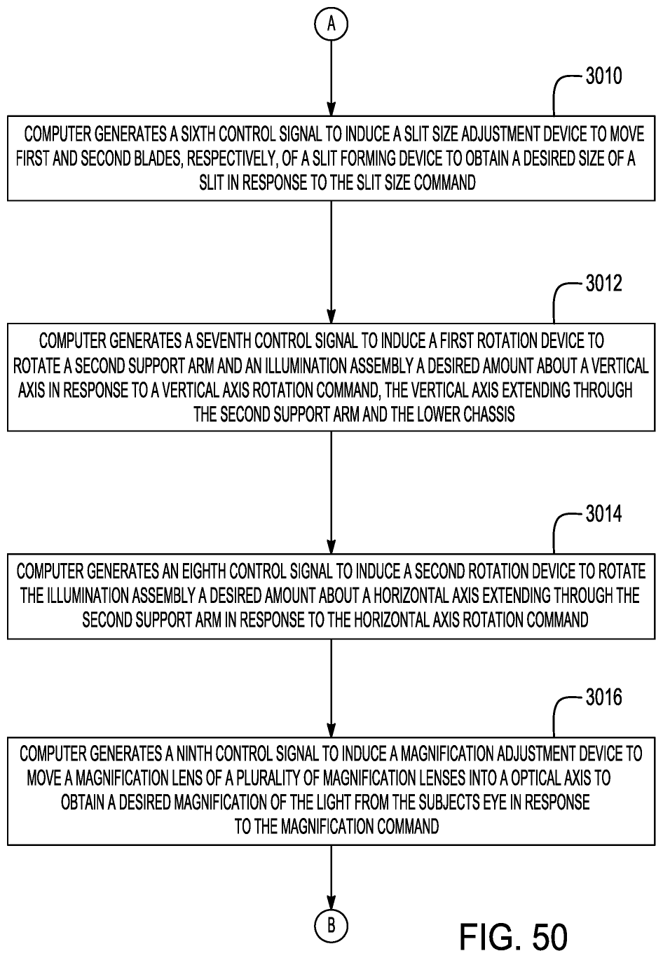

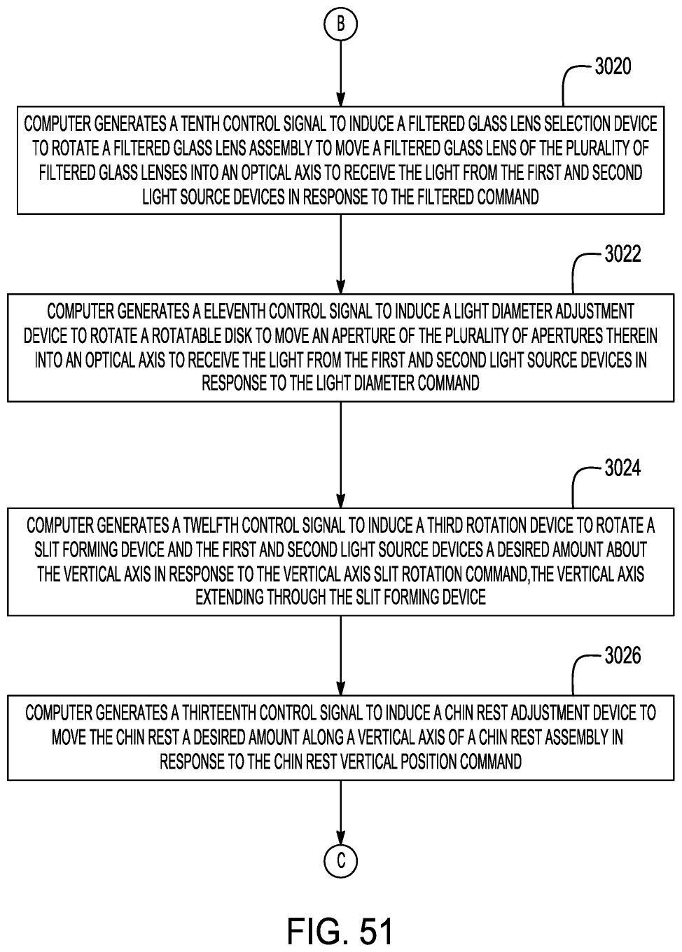

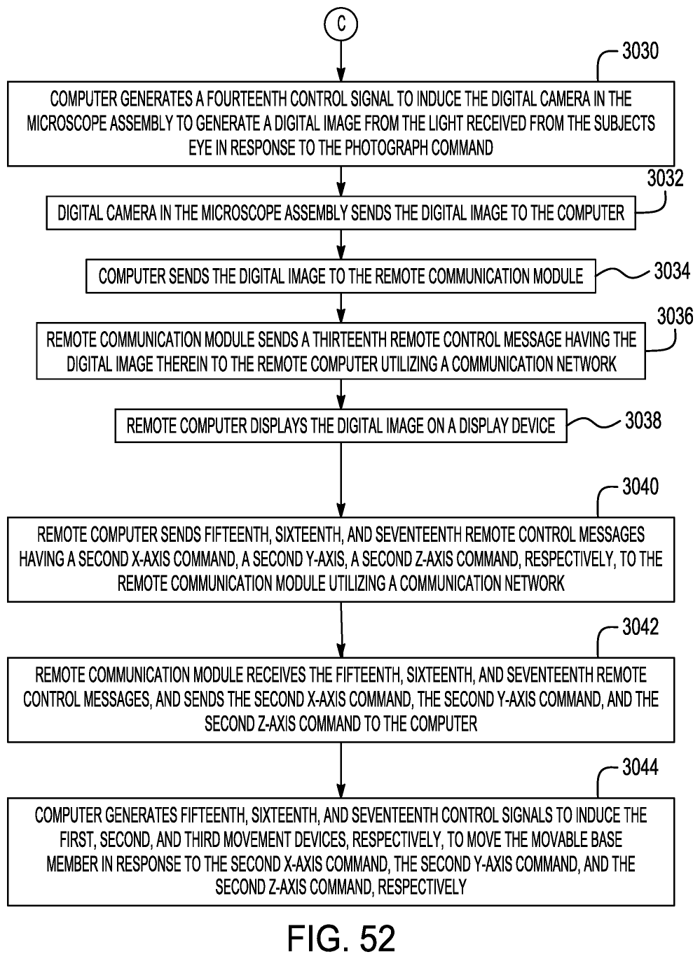

Referring to FIGS. 1, 3 and 49-52, a flowchart of a method for utilizing the slit-lamp microscope system 20 in accordance with another exemplary embodiment will now be explained.

At step 3000, the remote computer 32 transmits first, second, third, fourth, fifth, sixth, seventh, eighth, ninth, tenth, eleventh, twelfth, and thirteenth remote control messages having a first x-axis command, a first y-axis command, a first z-axis command, a light intensity command, a slit size command, a vertical axis rotation command, a horizontal axis rotation command, a magnification command, a filter command, a light diameter command, and a vertical axis slit rotation command, a chin rest vertical position command, and a photograph command, respectively. In an exemplary embodiment, the first x-axis command, the first y-axis command, the first z-axis command, the light intensity command, the slit size command, the vertical axis rotation command, the horizontal axis rotation command, the magnification command, the filter command, the light diameter command, the vertical axis slit rotation command, the chin rest vertical position command, and the photograph command correspond to respective user inputs that are transmitted from the input device 33 (shown in FIG. 3) to the remote computer 32.

At step 3002, the remote communication module 100 receives the first, second, third, fourth, fifth, sixth, seventh, eighth, ninth, tenth, eleventh, twelfth, and thirteenth remote control messages, and sends the first x-axis command, the first y-axis command, the first z-axis command, the light intensity command, the slit size command, the vertical axis rotation command, the horizontal axis rotation command, the magnification command, the filter command, the light diameter command, the vertical axis slit rotation command, the chin rest vertical position command, and the photograph command to a computer 90.

At step 3004, the computer 90 generates first, second, and third control signals to induce first, second, and third movement devices 51, 52, 53, respectively, to move a movable base member 44 in response to the first x-axis command, the first y-axis command, and the first z-axis command, respectively.

At step 3006, the computer 90 generates a fourth control signal to induce a first light intensity adjustment device 652 to adjust an intensity of the light being emitted from a first light source device 654 a desired amount in response to the light intensity command.

At step 3008, the computer 90 generates a fifth control signal to induce a second light intensity adjustment device 656 to adjust an intensity of the light being emitted from a second light source device 658 a desired amount in response to the light intensity command.

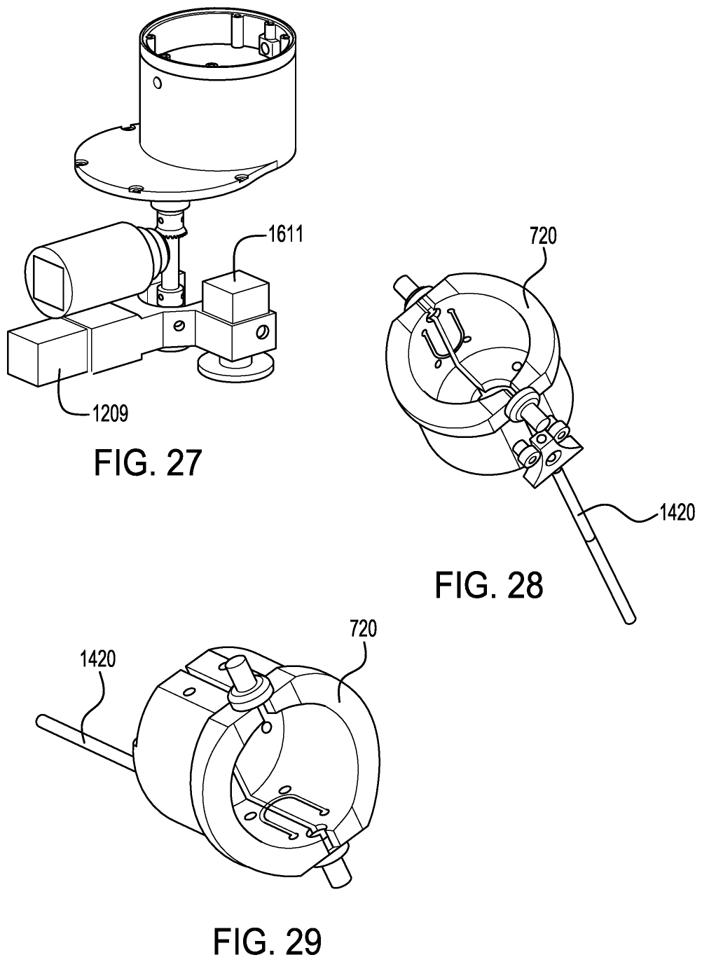

At step 3010, the computer 90 generates a sixth control signal to induce a slit size adjustment device 730 to move first and second blades 1331, 1332 (shown in FIGS. 27-36), respectively, of a slit forming device 720 to obtain a desired size of a slit in response to the slit size command.

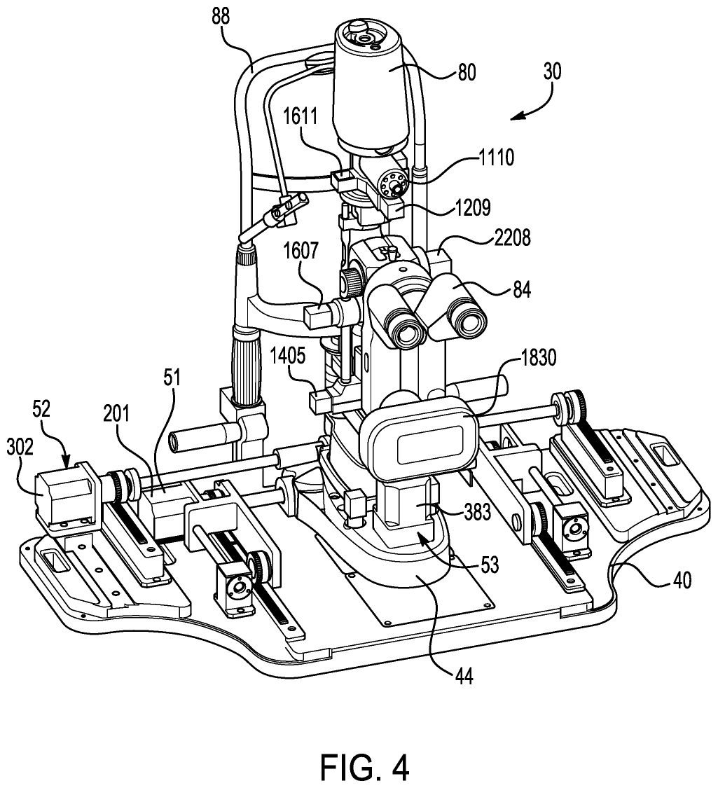

At step 3012, the computer 90 generates a seventh control signal to induce a first rotation device 744 (shown in FIG. 22) to rotate a second support arm 72 (shown in FIG. 4) and an illumination assembly 80 a desired amount about a vertical axis 754 (shown in FIG. 22) in response to the vertical axis rotation command. The vertical axis 754 extends through the second support arm 72 and the lower chassis 60.

At step 3014, the computer 90 generates an eighth control signal to induce a second rotation device 750 (shown in FIG. 23) to rotate the illumination assembly 80 a desired amount about a horizontal axis 751 (shown in FIG. 41) extending through the second support arm 72 in response to the horizontal axis rotation command.

At step 3016, the computer 90 generates a ninth control signal to induce a magnification adjustment device 2010 to move a magnification lens of a plurality of magnification lenses 2000 (shown in FIG. 13) into an optical axis 782 (shown in FIG. 13) to obtain a desired magnification of the light from the subjects eye 81 (shown in FIG. 13) in response to the magnification command.

At step 3020, the computer 90 generates a tenth control signal to induce a filtered glass lens selection device 702 to rotate a filtered glass lens assembly 700 (shown in FIG. 43) to move a filtered glass lens of the plurality of filtered glass lenses 1152 (shown in FIG. 43) into an optical axis 781 (shown in FIG. 13) to receive the light from the first and second light source devices 654, 658 in response to the filter command.

At step 3022, the computer 90 generates an eleventh control signal to induce a light diameter adjustment device 688 to rotate a rotatable disk 686 (shown in FIG. 43) to move an aperture of the plurality of apertures 1070 therein into an optical axis 781 (shown in FIG. 13) to receive light from the first and second light source devices 654, 658 in response to the light diameter command.

At step 3024, the computer 90 generates a twelfth control signal to induce a third rotation device 760 to rotate a slit forming device 720 (shown in FIGS. 27-36) and the first and second light source devices 654, 658 a desired amount about the vertical axis 1670 (shown in FIG. 44) in response to the vertical axis slit rotation command. The vertical axis 1670 extends through the slit forming device 720.

At step 3026, the computer 90 generates a thirteenth control signal to induce a chin rest adjustment device 2350 to move the chin rest 2324 a desired amount along a vertical axis 2351 (shown in FIG. 48) of a chin rest assembly 88 (shown in FIG. 47) in response to the chin rest vertical position command.

At step 3030, the computer 90 generates a fourteenth control signal to induce the digital camera 1830 (shown in FIG. 7) in the microscope assembly 84 to generate a digital image from the light received from the subjects eye 81 (shown in FIG. 13) in response to the photograph command.

At step 3032, the digital camera 1830 in the microscope assembly 84 sends the digital image to the computer 90.

At step 3034, the computer 90 sends the digital image to the remote communication module 100 (shown in FIG. 3).

At step 3036, the remote communication module 100 sends a thirteenth remote control message having the digital image therein to the remote computer 32 utilizing the communication network 35.

At step 3038, the remote computer 32 displays the digital image on a display device 34.

At step 3040, the remote computer 32 sends fifteenth, sixteenth, and seventeenth remote control messages having a second x-axis command, a second y-axis command, a second z-axis command, respectively to the remote communication module 100 utilizing the communication network 35.

At step 3042, the remote communication module 100 receives the fifteenth, sixteenth, and seventeenth remote control messages, and sends the second x-axis command, the second y-axis command, and the second z-axis command to the computer 90.

At step 3044, the computer 90 generates fifteenth, sixteenth, and seventeenth control signals to induce the first, second, and third movement devices 51, 52, 53, respectively, to move the movable base member 44 in response to the second x-axis command, the second y-axis command, and the second z-axis command, respectively.

The slit-lamp microscope system described herein provides a substantial advantage over other systems. In particular, the slit-lamp microscope system includes a slit-lamp microscope with first, second, and third movement devices that can move a movable base member parallel to an x-axis, a y-axis, and a z-axis, respectively in response to remote control messages from a received by a remote communication module in the slit-lamp microscope. Further, the slit-lamp microscope has a slit size adjustment device that moves first and second blades, respectively of a slit forming device in response to a remote control message received by the remote communication module in the slit-lamp microscope.

While the claimed invention has been described in detail in connection with only a limited number of embodiments, it should be readily understood that the invention is not limited to such disclosed embodiments. Rather, the claimed invention can be modified to incorporate any number of variations, alterations, substitutions or equivalent arrangements not heretofore described, but which are commensurate with the spirit and scope of the invention. Additionally, while various embodiments of the claimed invention have been described, it is to be understood that aspects of the invention may include only some of the described embodiments. Accordingly, the claimed invention is not to be seen as limited by the foregoing description.

What is claimed is:

1. A slit-lamp microscope, comprising:
a base plate defining an x-axis and a z-axis;
a movable base member slidably disposed on the base plate;
a first movement device operably coupled to the base plate and the movable base member, the first movement device moving the movable base member left and right parallel to the x-axis;
a second movement device operably coupled to the base plate and the movable base member, the second movement device moving the movable base member forward and backward parallel to the z-axis;
a lower chassis operably coupled to the movable base member;
a first support arm being coupled to the lower chassis and to a microscope assembly;
a second support arm being coupled to the lower chassis and an illumination assembly;
the illumination assembly having a slit forming device and a light source device, the slit forming device having a slit for receiving light therethrough that is output from the light source device, and projects the light from the slit forming device onto a subjects eye;
the microscope assembly receiving light from the subjects eye, the microscope assembly having a digital camera therein;
a computer operably coupled to the first, second, and third movement devices, the microscope assembly, and a remote communication module;
the remote communication module receiving first, second, and third remote control messages with a first x-axis command, a first z-axis command, and a photograph command, respectively, and the remote communication module sending the first x-axis command, the first z-axis command, and the photograph command to the computer;
the computer generating the first and second control signals to induce the first and second movement devices, respectively, to move the movable base member in response to the first x-axis command and the first z-axis command, respectively; and
the computer generating a third control signal to induce the digital camera of the microscope assembly to generate a digital image from the light received from the subjects eye in response to the photograph command.

2. The slit-lamp microscope of claim 1, wherein:
the base plate further defining a y-axis, the y-axis being perpendicular to the x-axis and the z-axis;
a third movement device operably coupled to the movable base member, the base plate, and the lower chassis; the third movement device moving the lower chassis upwardly and downwardly parallel to the y-axis;
the remote communication module receiving a fourth remote control message with a first y-axis command, and the remote communication module sending the first y-axis command to the computer; and
the computer generating a fourth control signal to induce the third movement device to move the movable base member in response to the first y-axis command.

3. The slit-lamp microscope of claim 2, wherein:
the remote communication module receiving fifth, sixth, and seventh remote control messages with a second x-axis command, a second y-axis command, and a second z-axis command, respectively, and the remote communication module sending the second x-axis command, the second y-axis command, the second z-axis command to the computer; and
the computer generating fifth, sixth, and seventh control signals to induce the first, second, and third movement devices, respectively, to move the movable base member in response to the second x-axis command, the second y-axis command, and the second z-axis command, respectively.

4. The slit-lamp microscope of claim 2, wherein:
the illumination assembly further includes a light intensity adjustment device operably coupled to the light source device;
the remote communication module receiving a fifth remote control message having a light intensity command, and the remote communication module sending the light intensity command to the computer; and
the computer generating a fifth control signal to induce the light intensity adjustment device to adjust an intensity of the light being emitted from the light source device in response to the light intensity command.

5. The slit-lamp microscope of claim 2, wherein:
the illumination assembly further includes a slit size adjustment device operably coupled to the slit forming device;
the remote communication module receiving a fifth remote control message having a slit size command, and the remote communication module sending the slit size command to the computer; and
the computer generating a fifth control signal to induce the slit size adjustment device to move first and second blades, respectively, of the slit forming device to obtain a desired size of the slit in response to the slit size command.

6. The slit-lamp microscope of claim 2, wherein:
the illumination assembly further includes a first rotation device operably coupled to the second support arm and the lower chassis, a vertical axis extending through the second support arm and the lower chassis;
the remote communication module receiving a fifth remote control message having a vertical axis rotation command, and the remote communication module sending the vertical axis rotation command to the computer; and
the computer generating a fifth control signal to induce the first rotation device to rotate the second support arm and the illumination assembly a desired amount about the vertical axis in response to the vertical axis rotation command.

7. The slit-lamp microscope of claim 2, wherein:
the illumination assembly further includes a first rotation device operably coupled to the second support arm, the second support arm having a horizontal axis extending therethrough;
the remote communication module receiving a fifth remote control message having a horizontal axis rotation command, and the remote communication module sending the horizontal axis rotation command to the computer; and
the computer generating a fifth control signal to induce the first rotation device to rotate the illumination assembly a desired amount about the horizontal axis in response to the horizontal axis rotation command.

8. The slit-lamp microscope of claim 2, wherein:
the microscope assembly further includes a magnification adjustment device that is operably coupled to a magnification lens assembly therein, the magnification lens assembly having a plurality of magnification lenses that magnify the light from the subjects eye;
the remote communication module receiving a fifth remote control message having a magnification command, and the remote communication module sending the magnification command to the computer; and
the computer generating a fifth control signal to induce the magnification adjustment device to move a magnification lens of the plurality of magnification lenses into an optical axis to adjust a magnification of the light from the subjects eye in response to the magnification command.

9. The slit-lamp microscope of claim 2, wherein:
the illumination assembly further includes a filtered glass lens selection device operably coupled to a filtered glass lens assembly, the filtered glass lens assembly having a plurality of filtered glass lenses;
the remote communication module receiving a fifth remote control message having a filter command, and the remote communication module sending the filter command to the computer; and
the computer generating a fifth control signal to induce the filtered glass lens selection device to rotate the filtered glass lens assembly to move a filtered glass lens of the plurality of filtered glass lenses into an optical axis to receive the light from the light source device in response to the filter command.

10. The slit-lamp microscope of claim 2, wherein:
the illumination assembly further includes a light diameter adjustment device operably coupled to a rotatable disk, the rotatable disk having a plurality of apertures therein each having a respective diameter, the rotatable disk receiving the light from the light source device;
the remote communication module receiving a fifth remote control message having a light diameter command, and the remote communication module sending the light diameter command to the computer; and
the computer generating a fifth control signal to induce the light diameter adjustment device to rotate the rotatable disk to move an aperture of the plurality of apertures into an optical axis to receive light from the light source device in response to the light diameter command.

11. The slit-lamp microscope of claim 2, wherein:
the slit forming device and the light source device having a vertical axis extending therethrough;
the illumination assembly further includes a first rotation device operably coupled to the second support arm that rotates the slit forming device and the light source device;
the remote communication module receiving a fifth remote control message having a vertical slit axis rotation command, and the remote communication module sending the vertical axis slit rotation command to the computer; and
the computer generating a fifth control signal to induce the first rotation device to rotate the slit forming device and the light source device a desired amount about the vertical axis in response to the vertical axis slit rotation command.

12. The slit-lamp microscope of claim 2, further comprising:
a chin rest assembly coupled to the base plate, the chin rest assembly having a vertical axis extending therethrough;
the remote communication module receiving a fifth remote control message having a chin rest vertical position command, and the remote communication module sending the chin rest vertical position command to the computer; and
the computer generating a fifth control signal to induce a chin rest adjustment device to move the chin rest a desired amount parallel to the vertical axis in response to the chin rest vertical position command.

13. A slit-lamp microscope, comprising:
a base plate;
a movable base member slidably disposed on the base plate;
a lower chassis operably coupled to the movable base member;
a first support arm being coupled to the lower chassis and to a microscope assembly;
a second support arm being coupled to the lower chassis and an illumination assembly;
the illumination assembly having a slit forming device, a light source device, and a slit size adjustment device operably coupled to the slit forming device; the slit forming device having a slit for receiving output from the light source device, and projects the light from the slit forming device onto a subjects eye;
the microscope assembly receiving light from the subjects eye, the microscope assembly having a digital camera therein;
a computer operably coupled to the slit size adjustment device, the microscope assembly, and a remote communication module;
the remote communication module receiving first and second remote control messages having a slit size command and a photograph command, respectively, and the remote communication module sending the slit size command and the photograph command to the computer;
the computer generating a first control signal to induce the slit size adjustment device to move first and second blades, respectively, of the slit forming device to obtain a desired size of the slit in response to the slit size command; and
the computer generating a second control signal to induce the digital camera in the microscope assembly to generate a digital image from the light received from the subjects eye in response to the photograph command.
